(19) United States
(12) Patent Application Publication
(10) Pub. No.: US 2014/0249051 A1
(43) Pub. Date: Sep. 4, 2014

(54) METHOD AND SYSTEM FOR ABO ANTIBODY DETECTION AND CHARACTERIZATION

(71) Applicants: Todd Lambert Lowary, Edmonton (CA); Christopher Warren Cairo, Edmonton (CA); Lori Jeanne West, Edmonton (CA); Jillian Mary Buriak, Edmonton (CA); Peter John Meloncelli, Edmonton (CA); Mylvaganam Jeyakanthan, Edmonton (CA); Anne Margaret Slaney, Spruce Grove (CA)

(72) Inventors: Todd Lambert Lowary, Edmonton (CA); Christopher Warren Cairo, Edmonton (CA); Lori Jeanne West, Edmonton (CA); Jillian Mary Buriak, Edmonton (CA); Peter John Meloncelli, Edmonton (CA); Mylvaganam Jeyakanthan, Edmonton (CA); Anne Margaret Slaney, Spruce Grove (CA)

(73) Assignee: THE GOVERNORS OF THE UNIVERSITY OF ALBERTA, Edmonton, AB (CA)

(21) Appl. No.: 14/342,255

(22) PCT Filed: Aug. 30, 2012
(86) PCT No.: PCT/CA2012/050602

§ 371 Date: May 16, 2014

Related U.S. Application Data
(60) Provisional application No. 61/529,082, filed on Aug. 30, 2011.

(51) Int. Cl. G01N 33/68

(52) U.S. Cl. CPC G01N 33/6854; G01N 2400/00
USPC 506/9

(57) ABSTRACT

The present application discloses a system and method for ABO antibody detection and characterization that can provide an alternative means for assessment and management of ABO-incompatible and ABO-compatible transplants. The method and system comprises determining an anti-ABO blood group antigen subtype antibody profile of a subject using a biological sample from the subject. The method and system can be used to evaluate the suitability of a donor blood or tissue product for a recipient subject by comparing the determined anti-ABO antigen subtype antibody profile of the recipient subject with the ABO histo-blood group or ABO histo-blood subgroup of a donor blood or tissue product. In order to define the subject's ABO histo-blood subgroup, the determined antibody profile is compared to known ABO histo-blood group antigen subtype profiles and/or known anti-ABO antigen subtype antibody profiles for ABO histo-blood subgroups to identify the ABO histo-blood subgroup of the subject. Profiles can be established by applying a sample to an array of surface-bound ABO antigens selected from the group of type I to type VI antigens of each blood group A, B or H.

### CROSS-REFERENCE TO RELATED APPLICATION

This application claims priority to and the benefit of U.S. Provisional Patent Application No. 61/529,082, filed Aug. 30, 2011, which is incorporated herein by reference in its entirety.

### FIELD OF THE INVENTION

The present application relates to a method to identify antibodies against ‘ABO blood group’ antigen subtypes. More specifically, the present application pertains to a method for detecting and characterizing anti-ABO antigen subtype antibodies, which can be useful, for example, in the fields of organ and cell/islet transplantation and blood transfusion. The method can use a glycan array to enable simultaneous detection and precise assessment of antibodies towards multiple ABO antigen subtypes.

### BACKGROUND OF THE INVENTION

In the clinical environment, ABO-compatibility between donor and recipient for organ (and cell) transplantation and blood transfusion is typically considered essential to avoid antibody-mediated graft rejection and/or hemolysis. The antibodies that can cause these destructive processes are pre-existing ‘natural’ antibodies that humans produce to ABO antigens not expressed in their own tissues. The ABO histo-blood group antigens are glycan structures expressed normally on many cells and tissues, schematically represented in FIG. 1. In general, determination of an individual's ABO blood type and assessment of their production of antibodies to ABO antigens has focused on the terminal antigen residues (FIG. 1). However, the ABO antigens are always found as part of a larger glycan structure, and the adjacent residues allow the carbohydrate antigens to be classed into subtypes. Each of the three major ABO antigens is present as one of six subtypes that are denoted in Table 1.

TABLE 1
ABO histo-blood group antigen subtypes.

| Type | Carbohydrate |
|---|---|
| Type I | ABO-β-(1→3)-β-D-Glcp NAc |
| Type II | ABO-β-(1→4)-β-D-Glcp NAc |
| Type III | ABO-β-(1→3)-α-D-Galp NAc |
| Type IV | ABO-β-(1→3)-β-D-Galp NAc |
| Type V | ABO-β-(1→3)-β-D-Galp |
| Type VI | ABO-β-(1→4)-β-D-Glcp |

These ABO antigen subtypes are differentially expressed on tissue and erythrocyte surfaces. (Ravn, V, Dabelsteen, E., APMIS, 108:1-28) (Oriol, R., Transplant. Proc., 1987, 19:4416-4420) Of the antigen subtypes, all except the type V antigen have been isolated from human sources. (Yamamoto, F., Immunohematology, 2004, 20:3-22) Considering that each of the three major ABO antigens (A, B and ‘H’) is present as any one of the six subtypes, there are a total of 18 major histo-blood group antigen subtypes in the ABO system.

In addition to antigen subtypes, the ABO system classifies individuals into subgroups. The ABO subgroups can be critical for the transplant and transfusion compatibility, with the A₁ and A₂ subgroups considered the best known examples of ABO subgroup differences. These subgroups are generated by two different N-acetylgalactosaminyl transferase (GTA) enzyme isoforms responsible for the biosynthesis of the antigens in both erythroblastserythrocytes and tissues. In the case of A₁ and A₂ subgroups, the enzymes GTA₁ and GTA₂ are responsible for the conversion of H antigen structures into A antigen structures. Antigens in these two subgroups differ both qualitatively and quantitatively. In the case of A₁ individuals, a greater density of type A histo-blood group antigens is present on their erythrocyte and tissue surfaces. In addition, it has been shown that A₁ and A₂ individuals express differing ratios of the ABO subtypes. (Clausen, H., Levery, S. B., Nudelman, E., et. al., Proc. Natl. Acad. Sci. U.S.A., 1985, 82:1199-1203) In the case of A₂ individuals, A type III and A type IV antigens are typically present in low levels compared to A type I and A type II antigens. This can sometimes cause lack of clarity when it comes to blood typing of patients. For example, approximately 8% of A₂ individuals and up to 35% of A₂B individuals have some anti-A₁ antibodies present, which, therefore, can complicate differentiation. (Sapanara, N. L., Swami, V, Besa, E. C., Laboratory Medicine, 2004, 35:538-541) A₂ individuals can usually be identified by additional testing due to the relatively common nature of this subgroup, however, 1% of type A individuals possess rare phenotypes which can result in discrepancies that are difficult to detect and may result in mis-typing (Sapanara, N. L., Swami, V, Besa, E. C., Laboratory Medicine, 2004, 35:538-541).

ABO Compatibility in Organ Transplantation

Although ABO-compatibility between donor and recipient is generally required for successful organ transplantation, intentional transplantation of an organ from an ABO-incompatible donor may be contemplated when an ABO-compatible donor cannot be located in a timely manner. Currently, prior to considering or planning an ABO-incompatible transplant, antibody levels in the recipient are measured using purchased reagent erythrocytes of the donor's blood type in an agglutination test (the current ‘gold standard’ test), which provides no guidance on ABO subgroup or subtype reactivity. As different tissues express different ABO antigen subtypes and different epitope densities, basing transplantation decisions on the presence or absence, as well as quantities and isotypes, of antibodies towards these antigen subtypes would offer a more accurate way of assessing and characterizing donor-recipient compatibility and incompatibility. Intentional ABO-incompatibilities of varying risk may be undertaken if finer resolution can be obtained, allowing more precise understanding of specific antigen matches and mismatches between donor and recipient. This would allow more efficient use of rare donor organs. Not only has donor-recipient ABO antigen subtype compatibility and its relation to ABO subgroups not been reported, but the variation of antibody levels and isotypes towards specific ABO antigen subtypes in human serum has also not been reported. There is presently no method or system available for the simultaneous measurement of antibodies towards each of the 18 ABO antigen subtypes and multiple antibody isotypes (IgG IgM and IgA). Such a method would be critical for improved precision in determination of donor-recipient compatibility, or precise degree and nature of incompatibility, in organ transplantation (and blood transfusion).

In addition to considering ABO antigen subtype, consideration must also be given to an individual's ABO subgroup. In the case of ABO-compatible transplantation, careful matching of donor and recipient ABO subgroup is vital to avoid accidental subgroup incompatibilities. (Gorodzinksy, F. P., Stechison, M. T., Poon, A., Arbus, G. S., CMA Journal, 1981, 125:871-873) Current technology is insufficient for accurate assessment of the degree of ABO-incompatibility both in ABO-compatible transplants (ie, to avoid inadvertent incompatible transplants) and in intentional planned ABO-incompatible transplants. Quantitative assessment of antibody levels towards the various specific ABO antigen subtypes would enable clinical decisions to be based on precise data rather than a subjective qualitative assessment, as with the methods currently employed (erythrocyte agglutination techniques).

ABO Compatibility in Transfusion

In the clinical setting, prior to transfusion, ABO compatibility between donor and recipient is confirmed using traditional methods. Typically the blood type of the patient is confirmed using erythrocyte agglutination. The typing is determined based on both the antigens present on the recipient's erythrocytes (forward typing) and on the ABO antibodies present or absent in the recipient's serum (reverse typing). These results are then matched to an appropriate donor blood product. In this case only antibodies to the core ABO antigens (structures 1-3) used for typing are detected; antibodies towards the antigen subtypes are not tested, which can present problems in the case of patients with certain ABO subgroups. For example, transfusion of A₁ blood into an A₂ individual can cause serious complications (Contreras, M. Hazlehurst, G. R. and Armitage, S. E. Brit. J. Haematology 1983, 55: 657-663). Methods currently exist for the identification of A₁ and A₂ individuals, however, many other subgroups are known and they are not routinely examined due to lack of sufficient effective typing methods. In the clinic, ABO subgroup incompatibilities often present as unexpected serum reactions. (Shaz, B. H., Transfusion Medicine and Hemostasis, Elsevier, New York, 2009) To avoid or minimize these unexpected serum reactions, more precise testing and matching between recipient and blood product is required. This is quite often done through ‘trial and error’ and can lead to delays in treatment. (Chaudhari, C. N., Misra, R. N., Nagpal, A. K., MJAFI, 2008, 64:371-372) In emergency situations this delay can result in transfusing blood with minor mismatch problems. (Chaudhari, C. N., Misra, R. N., Nagpal, A. K., MJAFI, 2008, 64:371-372) The precise identification of the root cause of these unexpected serum reactions will enable more accurate matching of a blood product to an individual patient, and reduce the cost in time and health outcomes required for proper matching of blood products by ‘trial and error’.

U.S. Pat. No. 7,897,328 B2 discloses a bead-based assay for detection of antibodies towards ABO antigen subtypes in blood typing. This detection assay makes use of subtypes of microbeads, each coated with a different blood group antigen, in flow cytometry or Luminex™ technology to detect antibodies towards multiple blood group subtypes. However, the disclosed assay is limited to measuring only a few interactions simultaneously due to the requirement of non-overlapping fluorophoreschromophores for detection.

There remains a need for a method that allows for the quantitative assessment of ABO subtype-specific antibodies for clinical use in both transplantation and transfusion. Current techniques do not reveal the presence or quantity or nature (i.e, isotype, etc.) of antibodies to ABO antigen subtypes. In the absence of this information, physicians act on the side of ‘caution’, often rejecting potential donors in the interest of patient safety, which may prevent them from receiving life-saving transplants and safe transfusions.

### SUMMARY OF INVENTION

An object of the present application is to provide a system and method for precise and comprehensive ABO antibody detection and characterization. One purpose of the present system and method is to provide an improved means for detection, assessment and management of ABO-related compatibilities and incompatibilies between donors and recipients in the clinical settings of transplantation and transfusion. Ideally, the present method and system can provide improved accuracy and efficiency in donor and recipient matching pre-transplant and in planning intentional incompatible transplants with the greatest degree of safety. The present method and system further enables monitoring of a patient's antibodies post-transplant, to promptly identify early stages of rejection, thus enabling rapid clinical intervention to prevent or minimize destruction from antibody-mediated injury. The present method and system can also facilitate improved matching between recipient and donor blood products in the case of transfusion. Identification of unexpected transfusion reactions and rare ABO blood groups would reduce the number of cases where the incorrect blood product is provided to an individual.

According to one aspect of the present application, there is provided a method of identifying an ABO histo-blood antigen subtype antibody profile of a subject comprising: (a) identifying the presence or absence of antibodies specific for at least one ABO histo-blood group antigen subtype in a biological sample from said subject; and (b) using the information from step (a) to generate the ABO histo-blood group antibody profile of the subject, wherein step (a) optionally includes quantifying the amount of the at least one anti-ABO histo-blood group antigen subtype antibody in the biological sample. In one embodiment, step (a) includes identifying the presence or absence of antibodies specific for up to 18 ABO histo-blood group antigen subtypes in a biological sample from said subject. In a specific embodiment, the method additionally comprises the step of comparing the ABO histo-blood antigen subtype antibody profile of the subject with an ABO histo-blood group, ABO histo-blood subgroup or ABO histo-blood antigen subtype profile of a donor blood or tissue product.

In accordance with another aspect, there is provided a method of evaluating the suitability of a donor blood or tissue product for a recipient subject comprising: determining an anti-ABO antigen subtype antibody profile using a biological sample from said recipient subject; determining the ABO histo-blood group, ABO histo-blood subgroup or ABO histo-blood antigen subtype profile of the donor blood or tissue product; and determining the suitability of the donor blood or tissue product for donation to said recipient subject based on a comparison of the determined anti-ABO antigen subtype antibody profile of the recipient subject with the ABO histo-blood group, ABO histo-blood subgroup or ABO histo-blood antigen subtype profile of the donor blood or tissue product.

In accordance with another aspect, there is provided a method of monitoring a recipient subject for an adverse reaction following transfusion with a donor blood product or transplantation with a donor tissue, said method comprising: determining a first anti-ABO antigen subtype antibody profile using a first biological sample from said recipient subject; determining a second anti-ABO antigen subtype antibody profile using a second biological sample from said recipient subject, wherein the second; comparing the first anti-ABO antigen subtype antibody profile with the second anti-ABO antigen subtype antibody profile to identify a change in the anti-ABO antigen subtype antibody profile as an indicator of an adverse reaction; and, optionally, repeating these steps to continue monitoring the recipient subject for an adverse reaction.

In accordance with a specific aspect there is provided a method in which serum from an individual is analyzed using the proposed glycan microarray for antibodies towards all 18 of the ABO histo-blood antigen subtypes. Use of the glycan microarray allows detection and characterization of ABO antibodies by determination of antibody levels towards the ABO antigen subtypes, as well as qualitative determination of antibody isotypes. This, combined with the knowledge of ABO subgroups, can enable antibody discrepancies to be identified and characterized. Ideally, all available blood products should also be assessed using the glycan array. This is not essential, but it can help identify any discrepancies in typing the blood donor.

According to another aspect, there is provided a method of identifying the ABO histo-blood subgroup of a subject comprising: determining an anti-ABO histo-blood group antigen subtype antibody profile using a biological sample from said subject; and comparing the determined antibody profile to known ABO histo-blood group antigen subtype profiles and/or known anti-ABO antigen subtype antibody profiles for ABO histo-blood subgroups to identify the ABO histo-blood subgroup of the subject.

In accordance with another aspect, there is provided an assay kit for determining an anti-ABO antigen subtype antibody profile of a subject, said kit comprising: a glycan microarray or macroarray containing a plurality of immobilized ABO antigens; and instructions for use.

### BRIEF DESCRIPTION OF THE FIGURES

FIG. 1 depicts the ABO histo-blood group antigen glycan structures.

FIG. 2 depicts a schematic representation of ABO antibody analysis using a glycan microarray.

FIG. 3 depicts a schematic representation of a clinical scenario where a glycan array according to one embodiment of the present application can be used effectively before and after ABO-incompatible transplantation.

FIG. 4 depicts a schematic representation of a clinical scenario where a glycan array according to one embodiment of the present application can be used in identifying unexpected transfusion related issues.

FIG. 5 depicts a fluorescent image of an ABO-SAMA array and a corresponding schematic of the array; the array was incubated with plasma from a blood type O individual and bound antibodies were detected with anti-human IgM-DyLight649.

FIG. 6 depicts the results of analysis of IgG isotype serum antibodies from a blood group O individual.

FIG. 7 depicts the results of analysis of IgM isotype serum antibodies from a blood group O individual.

FIG. 8 depicts the results of analysis of IgG isotype serum antibodies from twenty blood group O infants who were transplanted with cardiac grafts from blood group A donors.

FIG. 9 depicts the results of analysis of IgM isotype antibodies from a blood group A₁ individual (‘secretor’).

FIG. 10 depicts the results of analysis of IgM isotype antibodies from a blood group A₁ individual (‘non-secretor’).

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, the term “ABO blood group,” refers to a class of human blood based on the inherited properties of erythrocytes as determined by the presence or absence of A or B histo-blood group antigens carried on the surface of erythrocytes. The four ABO blood groups are A, B, AB and O, which are characterized by the presence of A antigens, B antigens or A and B antigens or the absence of A and B antigens with the presence of the H antigen only, respectively, on the surface of erythrocytes.

As used herein, the term “ABO antigen subtype,” refers to subtypes of the A, B and H (O) antigens. As described above, the ABO histo-blood group antigens are glycan structures, which are always found as part of a larger glycan structure, and the adjacent residues allow the carbohydrate antigens to be classed into subtypes. Each of these ABO antigens is present as one of six subtypes that are denoted in Table 1 above. It is possible that additional subtypes will be identified in the future. The present method and system can be adapted to take advantage of information relating to the presence or absence, or the amount, of such yet to be identified subtypes.

As used herein, the term “ABO histo-blood antigen subtype profile,” refers to the ABO histo-blood antigen subtypes expressed on or in a tissue or organ. A profile can include information as to the presence or absense of at least one of the ABO histo-blood antigen subtypes, while a full profile can further include information as to the absolute or relative amounts of the ABO histo-blood antigen subtypes expressed on or in the tissue or organ.

As used herein, the term “anti-ABO antigen subtype antibody profile,” refers to the anti-ABO histo-blood group antigen subtype antibodies present in a sample or a subject. A basic profile includes information as to the presence or absence at least one of the anti-ABO antigen subtype antibodies, while a full profile can further include information as to the absolute or relative amounts of the antibodies present in a sample or a subject. The profile can further include information as to the isotypes of the anti-ABO antigen subtype antibodies present. In one example, the anti-ABO antigen subtype antibody profile includes information as to the presence or absence, or as to the absolute or relative amounts, and/or isotypes, of anti-ABO antigen subtype antibodies specific for more than one ABO histo-blood group antigen subtypes, such as more than one of the 18 major ABO histo-blood group antigen subtypes. In another example, the anti-ABO antigen subtype antibody profile includes information as to the presence or absence, or as to the absolute or relative amounts, of anti-ABO antigen subtype antibodies specific for the 18 major ABO histo-blood group antigen subtypes.

As used herein, the term “ABO subgroup,” is used to refer to a subgroup within the broad ABO blood group classification. These subgroups may be characterized by qualitative and quantitative differences in ABO antigen subtype profiles.

As used herein, the term “linker group,” refers to one or more bifunctional molecules that can be used to covalently couple a glycan antigen to the surface of a microarray, macroarray or protein. The linker group is attached to the glycan antigen in a position that does not affect or does not substantially diminish the antigenicity of the glycan.

As used herein, the term “biological sample,” refers to a blood or tissue derived sample that contains ABO subtype antigens or anti-ABO antigen subtype antibodies, which includes, but is not limited to: organ tissue and erythrocytes [ABO subtype antigens] and serum, plasma, blood (i.e., whole blood) [anti-ABO antigen subtype antibodies].

The method and system described herein provides a means for physicians and other clinicians to assess ABO-compatibility or -incompatability, accurately and, consequently, to allow transplantation or transfusion boundaries to be firmly established. Use of present method and system can reduce adverse reactions resulting from unintentional or accidental use of incompatible blood products and organstissuescells, and will assist in allowing intentional incompatibilities to be used safely, thus saving lives.

The present method and system takes advantage of the fact that ABO antigen subtypes are expressed in varying densities and ratios in different individuals and are differentially expressed on erythrocytes and in tissues. This has important implications for both transplantation and transfusion matching. In one embodiment, the present method comprises the use of a device having multiple ABO histo-blood group antigens coupled to a carrier, for example, a glycan micro- or macro-array. The device is then used to measure antibodies towards the ABO antigen subtypes qualitatively or quantitatively. This method is useful, for example, because it can allow for transplantation and transfusion compatibility to be assessed more accurately than current available methods allow.

In accordance with one aspect there is provided an immunological method for determining the anti-ABO antigen subtype antibody profile of a subject. The method comprises the step of determining the presence or absence of antibodies against ABO antigen subtypes within a biological sample obtained from a subject. Typically, the biological sample is blood, serum or plasma. In determining the anti-ABO antigen subtype antibody profile of the subject, it is advantageous to test for the presence or absence of antibodies against all of the ABO antigen subtypes.

The present method comprises contacting a biological sample from a subject with at least one ABO antigen subtype and detecting binding complexes formed from binding of the at least one ABO antigen subtype with the anti-ABO antigen subtypes antibodies present in the sample. The binding complexes are then detected in such a manner that the identity of the anti-ABO antigen subtype antibodies is determined. Optionally, the amount of each anti-ABO antigen subtype antibody is also determined. Preferably, the at least one ABO antigen subtype is immobilized on a support.

Immunoassays for detecting target antibodies of interest in samples can be either competitive or noncompetitive. In either case, the presence of a complex formed between the ABO antigen subtypes described above and antibodies specific therefor can be detected by any of the known methods common in the art, such as fluorescent antibody spectroscopy or colorimetry.

In accordance with one embodiment, the method is performed using ABO antigen subtypes that are immobilized on a solid surface, such as a microarray or macroarray in which the location of each ABO antigen subtype is known. This configuration facilitates simultaneous detection and, optionally, quantification and characterization (e.g., in terms of isotype), of the anti-ABO antigen subtype antibodies present in the biological sample.

ABO Matching in Transplantation

Improved matching in transplantation is achieved using the presently described method, by measuring antibodies towards the ABO histo-blood group antigen subtypes and using this information, combined with the knowledge of antigen subtype expression on the donor organ, to determine the degree of compatibility of such a transplant. A preferred platform, based on presently available techniques, for measuring these antibodies is a glycan microarray or macroarray, as this approach makes it possible not only to measure antibodies towards all 18 ABO antigen subtypes but also to identify different antibody isotypes in a quantitative manner.

Different cells and tissues express different ABO antigen subtypes in varying amounts and densities; this is determined genetically by various factors including the Le and Se gene (Oriol, R., Transplantation Proceedings, 1987, 4416-4420). While it is known that ABO antigen subtypes are expressed on various tissue surfaces, it is not presently appreciated by clinicians that ABO subtype expression can vary between different tissues in the same individual. Simply considering the ABO blood type of a donor, as is currently practiced in evaluating suitability of a donor tissue, would not provide any information regarding the ABO antigen subtypes expressed on the donor tissue.

In one embodiment of the present application, a patient can be assessed for suitability for an ABO-incompatible organ transplant using the ABO glycan array. The ABO antigen subtype expression on the potential donor organ would be determined. The patient would be assessed for antibodies towards the ABO antigen subtypes and this would be compared to the known expression of these antigen subtypes on the donor organ. Low antibody levels towards the ABO antigen subtypes present on the donor organ would indicate that the transplantation could be achieved successfully. Post-transplant, the patient would then be monitored for antibodies towards the ABO antigen subtypes present on the donor organ. Any development of antibodies or increased antibody quantity could be detected and appropriate clinical measures enacted. Following therapeutic interventions, the glycan array would be an invaluable tool for further monitoring of antibody re-accumulation or sustained deficiency over time.

In another embodiment, a patient can be assessed for suitability for an ABO-compatible transplant using this glycan array. In this case, the patient's serum antibodies are assessed to identify unexpected serum reactions potentially due to rare ABO subgroups. This assessment would be achieved using the glycan array, identifying and characterizing any antibodies present towards the 18 ABO antigen subtypes. It is important that there are only low antibody levels towards the ABO antigen subtypes present on the donor organ. In ABO-compatible transplantation, this analysis is less important than in the case of intentional ABO-incompatible transplantation. However, in rare cases unexpected antibody-mediated reactions do occur and are thought to contribute to chronic graft damage. Through this method many of these reactions can be identified beforehand and any risks minimized. Ideally, serum from the organ donor should also be assessed for antibody levels towards the ABO antigen subtypes. Although this is not essential, this approach helps to identify unexpected serum reactions, acting as a failsafe prior to transplantation.

ABO Matching in Transfusion

Improved matching in transfusion is achieved using the presently described method, by measuring serum antibodies of the recipient towards the ABO histo-blood group antigen subtypes in the recipient prior to transfusion. This information can then be used to aid in the precise identification and etiology of unexpected transfusion reactions and assist in identifying the most compatible blood products. In most cases these discrepancies are caused by rare ABO subgroups; therefore, the ability to identify or narrow down to a small number of rare subgroup choices would be of value to physicians. This will reduce the amount of ‘trial and error’ required to identify a compatible blood product. New subgroups continue to be identified. For example Pruss and co-workers reported a new A blood group variant (Aw11), and the patient who possessed this variant had anti-A antibody levels that were difficult to detect on multiple commercially available systems. (Pruss, A., Heymann, G A., Braun, J., et. al., Vox Sanguinis, 2006, 90:195-197) The presently described method would enable this discrepancy to be identified accurately and rapidly, and the patient provided with a compatible blood product.

Preparation of ABO Glycan Structures

As noted above, the ABO antigen subtypes are glycans. The synthesis of the glycans with the prerequisite alkene aglycone has been previously reported. (Meloncelli, P. J., Lowary, T. L., Carbohydr. Res., 2010, 345:2305-2322) (Meloncelli, P. J., Lowary, T. L., Aust. J. Chem., 2009, 62:558-574) (Meloncelli, P. J., West, L. J., Lowary, T. L., Carbohydr. Res., 2011, 346:1406-1426). To successfully achieve conjugation of these glycans to a solid surface, such as an array surface, modification is required.

In one embodiment, the alkene group in the glycan can be converted to a different functional group and then attached to a solid surface, such as an array surface, bearing groups that are reactive with this different functional group. In one specific embodiment, the alkene can be converted to an amine and then attached to a solid surface, such as an array surface, bearing amine-reactive functional groups including, but not limited to, an N-hydroxysuccinimide ester. The conversion of the alkene to the amine does not affect the three dimensional shape of the glycan and allows the glycan to retain its antigenicity so that it can be selectively bound by its anti-ABO antigen subtype antibody after immobilization.

In an alternative embodiment, a bifunctional linker is employed. Such a linker group will react with the glycans in without affecting the three dimensional shape of the glycan and, thereby, allow the glycan to retain its antigenicity so that it can be selectively bound by its anti-ABO antigen subtype antibody after immobilization.

In a specific embodiment, the linker group is an activated p-nitrophenyl ester linker (Scheme 1). In this embodiment, the ABO glycan 4 is photochemically reacted with cysteamine to provide the requisite amine linked glycan 5. This is then treated with the heterobifunctional linker 6 in N,N-dimethylacetamide (“DMA”) to provide the glycan with an activated ester linker 7 suitable for conjugating to amine functionalized surfaces. One skilled of the art will understand that many variations of this attachment chemistry are possible, and that this is an illustrative example that is not intended to be limiting. Experimental details for this chemistry are discussed in the Examples below.

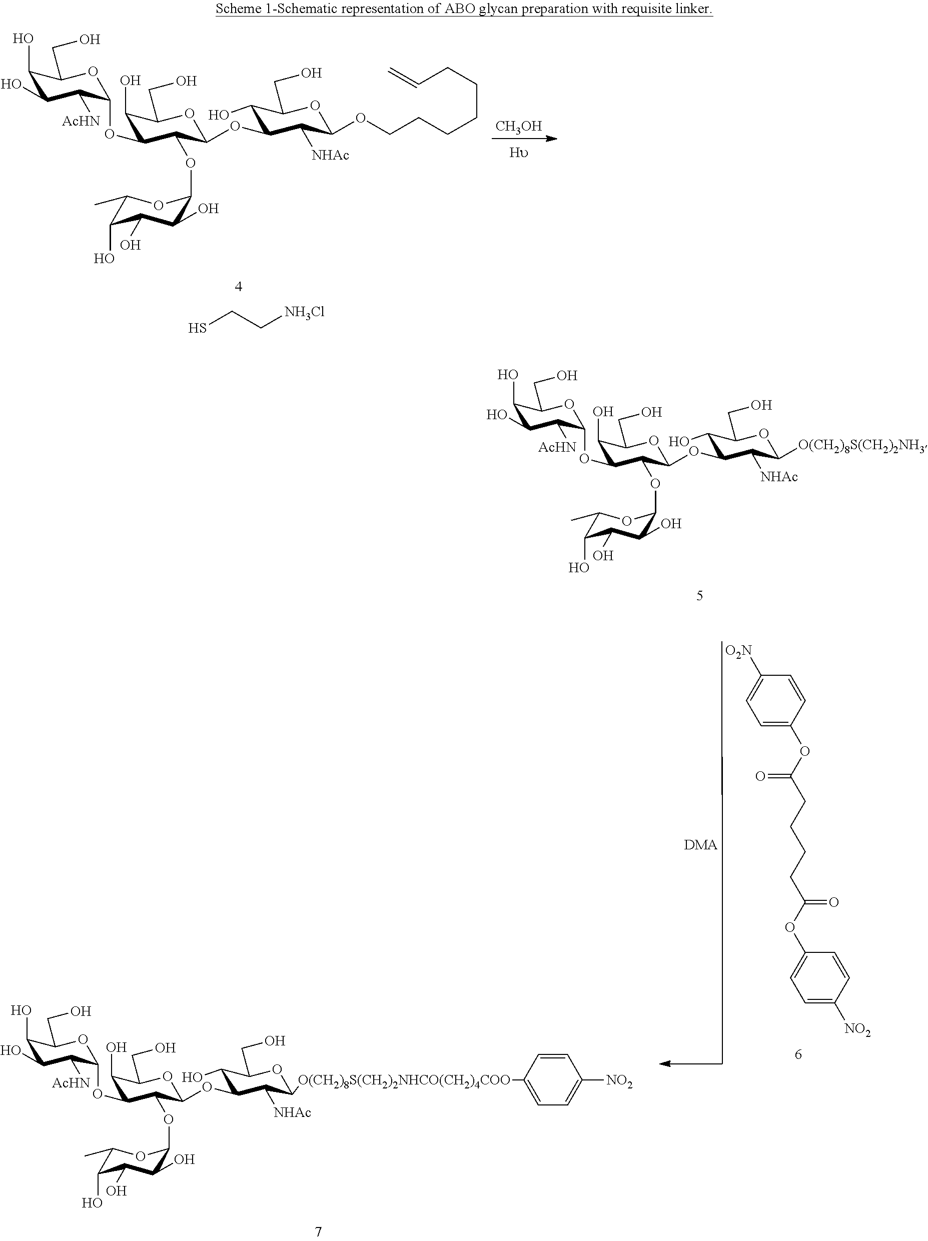

Conjugation to Glycan an Array Surface

Glycan arrays comprising a plurality of immobilized glycans have been fabricated using various methods. None of the previously fabricated arrays were designed to include a plurality of immobilized ABO subtype antigens as required for the present methods and systems. However, as would be readily appreciated by a worker skilled in the art, similar techiques can be used in manufacturing the glycan arrays of the present application. By way of example, glycan arrays have been prepared using biotinylated glycosides that are immobilized on a streptavidin coated plate (Bochner, B. S., Alvarez, R. A., Mehta, et. al., J. Biol. Chem. 2005, 280:4307-4312) and using glycans having a terminal amine that are immobilized on an amino-reactive N-hydroxysuccinimide-activated microglass surface (Blixt, O., Head, S., Mondala, T., et. al., Proc. Natl. Acad. Sci. U.S.A., 2004, 101:17033-17038).

In accordance with a specific, non-limiting embodiment, the ABO subtype antigen glycan is attached to a glass array surface using a pin microarray device. One skilled in the art will understand that there are many devices that can deposit glycans onto a surface and this example is not designed to be limiting. In one embodiment, at least one and as many as 18 ABO subtype antigens, functionalized to include a terminal amine (e.g., structure 5 in Scheme 1) can be attached to a glass slide bearing amine-reactive groups such as, but not limited to, an N-hydroxysuccinimide ester (Scheme 2). In this example the ABO glycan is prepared as a buffered solution in water. One skilled in the art will recognize that the can buffer enhance the efficiency of the attachment and is not essential.

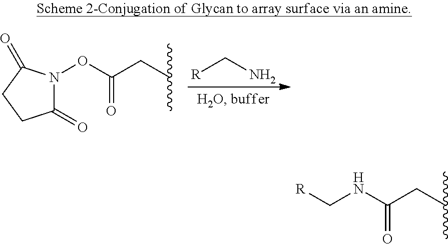

In another embodiment, at least one and as many as 18 ABO subtype antigens are printed onto the glycan array surface via a bifunctional linker using a commercially available microarray printer. In this example the ABO glycan is prepared as a solution in DMA (Scheme 3).

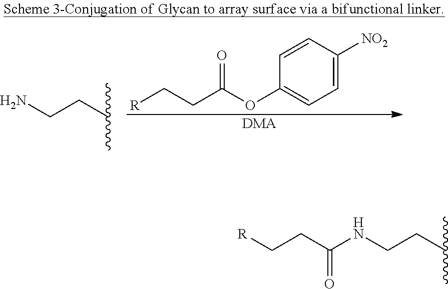

In another embodiment, at least one and as many as 18 ABO subtype antigens coupled to a protein such as bovine serum albumin (BSA) can be attached to a glass slide bearing amine-reactive groups such as, but not limited to, an epoxide (Scheme 4). In this example the ABO glycan-BSA conjugate is prepared as a buffered solution in water. One skilled in the art will recognize that the can buffer enhance the efficiency of the attachment and is not essential.

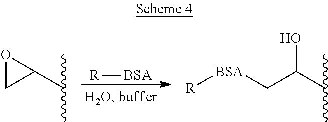

Other proteins that can be used in preparing a glycan-protein conjugate for immobilization include, but are not limited to, human serum albumin (HSA), keyhole limpet hemocyanin (KLH), chicken serum albumin (CSA), tetanus toxoid (TT) or diphtheria toxin mutant Cross-reactive material 197 Protein (CRM197).

In another embodiment, at least one and as many as 18 ABO subtype antigens coupled to biotin can be attached to a streptavidin-coated slide or plate. In this case, the biotinylated antigens are prepared using standard biotinylation techniques.

Method for Determining and Transplantation and Transfusion n Matching

In one embodiment of the invention, the method relies on a device that can simultaneously measure serum antibodies towards multiple ABO antigen subtypes. In addition, this device can simultaneously measure antibody isotypes (IgG IgM and IgA) against these antigens. One skilled in the art will understand that all of these antibodies can be simultaneously detected, however, this is not intended to limit the detection of only one or two of these antibody isotypes.

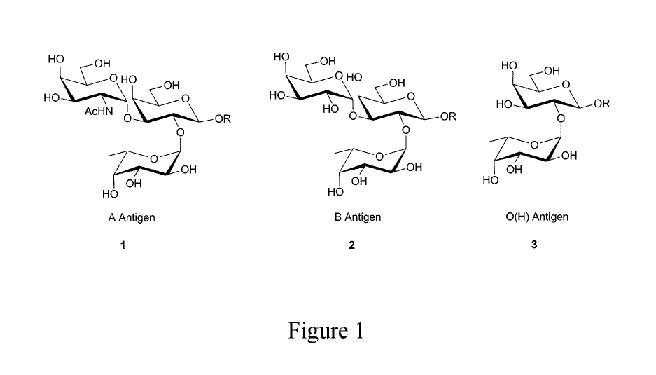

The chemical structures of the ABO histo-blood group antigens are shown in FIG. 1. The ABO blood group antigens can be further classified by the type of linkage connecting them to the remainder of the glycan motif. As shown in Table 1, six different families have been identified, termed type Ito type VI based on the monosaccharide residue and position to which the ‘reducing end’ O-galactose moiety is linked. All types are meant to be included within the scope of this invention as useful antigens for the detection and characterization of ABO antibodies and anti-ABO antigen subtype antibody profiles. Of course, one skilled in the art will appreciate that a smaller subset of these carbohydrate antigens can also typically be used.

In one embodiment, a glycan micro- or macro-array is used as the solid support for the ABO histo-blood group antigens. Other solid support can also be used and thus this description is not meant to be limiting. This glycan micro- or macro-array is typically, but not limited to, a chemically modified glass surface. One skilled in the art will understand that many variations in both surface and functionalization are possible.

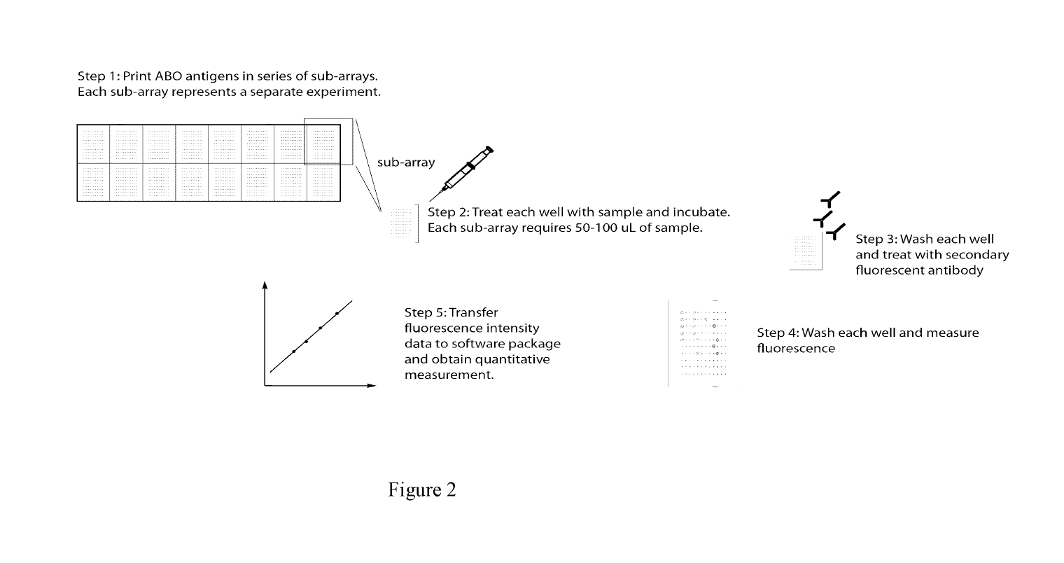

The method for determining serum antibody levels is presented in the General Methods section of this patent and schematically represented in FIG. 2. This schematic representation is not meant to be limiting; one skilled of the art will understand that variations of the above technique are possible. Detection of the bound serum antibody requires a secondary antibody with a label. The secondary antibody will bind to the bound serum antibody. For example, the secondary antibody can be an anti-human antibody (i.e., one that will recognize and bind to any human antibody) or it can be an antibody that has specficity for any human antibody of a specific isotype, which can be, but is not limited to, anti-human IgG (e.g., IgG1, IgG2, IgG3, IgG4), IgM, IgE, IgD and/or IgA antibodies or any combination of these antibodies.

The secondary antibody can be labeled using a label that is detectable by, for example, spectroscopic, photochemical, biochemical, immunochemical, electrical, optical, or chemical means. In one embodiment of the invention this label is a fluorophore. In another embodiment this label is a chromophore or chromophore-generating enzyme. In the case of a fluorescent indicator, detection can be achieved using a fluorescence array reader. In the case of a chromophore or chromophore-generating enzyme, any device capable of recording color intensities would be suitable, including, but not limited to, a flatbed scanner. One skilled of the art will realize that any number of labels and detection techniques are possible and that the above is not meant to be limiting.

In accordance with another aspect of the present application, there is provided an immunoassay kit that comprises one or more ABO antigen subtype glycans as described above and reagents for the detection of binding complexes formed with the ABO antigen subtype glycans and antibodies specific for the ABO antigen subtype glycans. The immunoassay kit can additionally include instructions for use and/or a container or other means for collecting a sample of bodily fluid from a subject suspected of having a bacterial infection.

In one embodiment, the immunoassay kit comprises the ABO antigen subtype glycans immobilized on a solid surface, such as the surface of a microarray or macroarray, for example as described above. Optionally, the micro- or macroarray includes a positive control. For example, the micro- or macroarray can include an immobilized antigen that is recognized by an antibody known to be present in all of the samples being tested. One example of such an antigen is the α-Gal epitope (α-Gal-(1→3)-β-Gal-(1→(3)4)-GlcNAc-R), which is found on glycolipids and glycoproteins of non-primate mammals and New World monkeys. (The α-Gal epitope and the anti-α-Gal antibody in xenotransplantation and in cancer immunotherapy. Galili U. Immunol Cell Biol., 2005, 83:674-86. Review) All humans are known to produce antibodies specific for α-Gal. By including this immobilized antigen on the immunoassay arrays, it is possible to confirm that the assay is functioning properly by noting the presence of signal indicating the formation of binding complexes at the location on the array where the control antigen is immobilized.

In an alternative embodiment, an immunoassay array having immobilized thereon antigens of a non-human origin can be used to evaluate a patient's eligibility for transplantation of a non-human organ.

Examples of ABO Cross-Matching in ABO-Compatible and Incompatible Transplantation

Scenarios for determining ABO serum antibody levels are presented in more detail in each of the Examples below. In transplantation, ABO-compatibility between donor and recipient is generally considered essential to prevent antibody-mediated rejection due to preformed ‘natural’ ABO antibodies. However, numerous transplants have been conducted across the ABO blood group barrier. The ABO antigen glycan array is ideally suited to identifying potential ABO-incompatible transplants with the greatest chance of success.

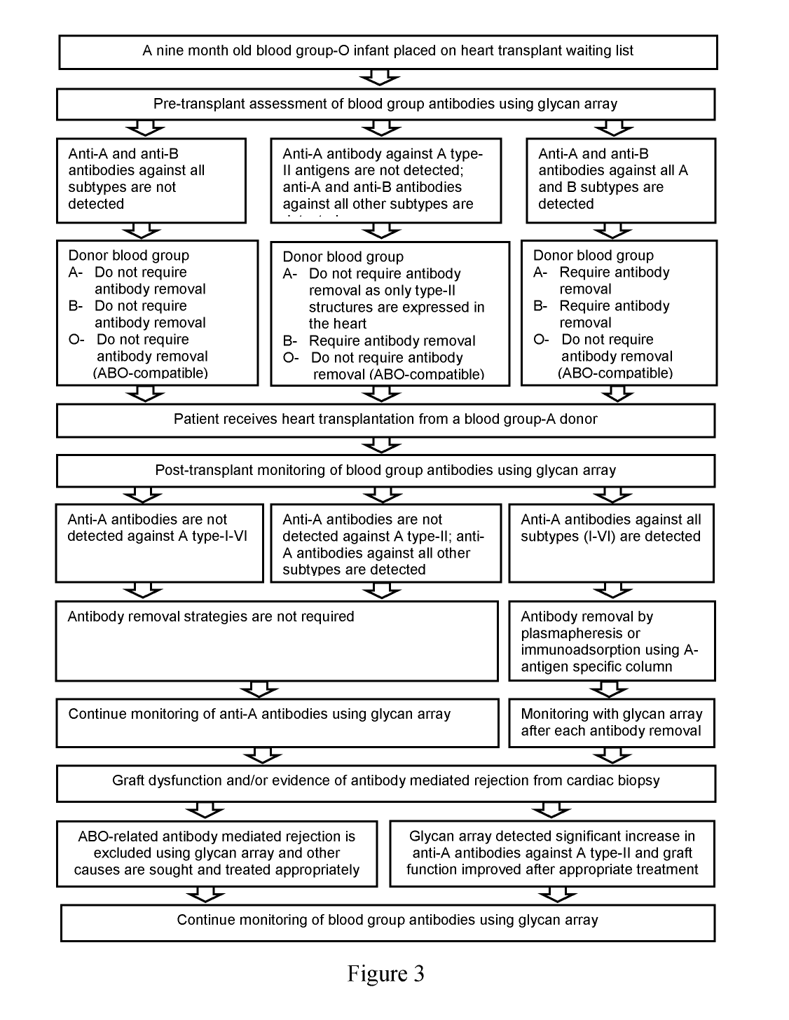

The example shown in FIG. 3, which is not intended to be limiting, highlights a clinical flowchart showing how the glycan array can be used effectively both before and after ABO-incompatible transplantation. In this example, a blood group-O infant requires a heart transplant. The patient is first screened using the glycan array to assess the ABO blood group antigen subtype specific antibody profile before transplant, and three different outcomes are depicted. In the first pathway, the patient would be able to receive either an ABO-incompatible (blood group-A or B or AB donor) or an ABO-compatible (blood group-O donor) heart transplant without the need for any antibody removal strategies. Similarly, the second pathway would not preclude the patient from receiving a blood group-A (ABO-incompatible) heart and not require antibody removal as the donor heart expresses only type II structures, although a blood group-B heart (ABO-incompatible) would involve antibody removal and lymphocyte and/or plasma cell depletion before and at the time of transplant, as well as possibly after transplant. If the patient had antibodies against all A and B subtypes (third pathway), an ABO-incompatible transplant would involve antibody removal and lymphocyte and/or plasma cell depletion before and at the time of transplant, as well as possibly after transplant. After the transplant, the patient is carefully monitored for signs of rejection in concert with the glycan array to measure ABO antibody levels. The array results (only three of many examples shown) direct clinicians toward the correct course of action; removal of antibodies or not, together with consideration of other interventions. In this case, elevated anti-A type II antibodies would be an early sign of impending heart graft rejection. This methodology enables these signs to be detected far earlier than with traditional techniques. Early clinical intervention greatly reduces the risk of rejection.

Example of ABO Matching in Transfusion

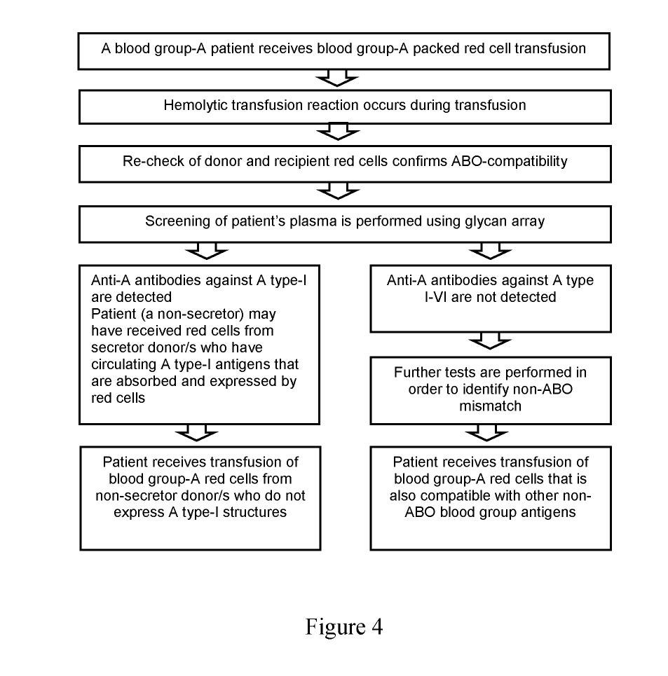

The example shown in FIG. 4, which is not intended to be limiting, highlights the method for improving ABO matching for a transfusion. In this case, a blood group-A patient who develops a reaction to an ABO-compatible transfusion has a sample of blood taken and serum antibodies towards the ABO histo-blood group antigen subtypes are measured via the glycan array described herein. If antibodies against A type I are detected (first pathway), the cause of the reaction may be identified quickly and a transfusion of more suitable blood product can commence. If no anti-A antibodies are detected (second pathway), the reaction likely originated from a non-ABO mismatch. Use of the glycan array to quickly rule out ABO antibodies as the cause for transfusion reactions will greatly reduce the time required to discover the actual cause and allow the patient to receive the correct blood product and other appropriate interventions.

To gain a better understanding of the invention described herein, the following examples are set forth. It should be understood that these examples are for illustrative purposes only. Therefore, they should not limit the scope of this invention in any way.

### EXAMPLES

### General Methods

Synthesis of ABO Glycans

The synthesis of the glycans with the prerequisite alkene aglycone was achieved using methods previously reported. (Meloncelli, P. J., Lowary, T. L., Carbohydr. Res., 2010, 345:2305-2322) (Meloncelli, P. J., Lowary, T. L., Aust. J. Chem., 2009, 62:558-574) (Meloncelli, P. J., West, L. J., Lowary, T. L., Carbohydr. Res., 2011, 346:1406-1426) A general method for the synthesis of the glycans with the activated ester linker is shown below. Yields for each of the steps are included in Table 2.

TABLE 2
Yields for reactions introducing the amine and PNP
linker onto the ABO antigens.

| | A Histo-Blood | | B Histo-Blood | | H Histo-Blood | |
|---|---|---|---|---|---|---|
| | Group | | Group | | Group | |
| | Amine | PNP | Amine | PNP | Amine | PNP |
| Type I | 91% | 7, 75% | 97% | 13, 83% | 97% | 19, 75% |
| Type II | 79% | 8, 79% | 97% | 14, 75% | 80% | 20, 78% |
| Type III | 93% | 9, 72% | 95% | 15, 92% | 98% | 21, 91% |
| Type IV | 96% | 10, 85% | 95% | 16, 78% | 95% | 22, 77% |
| Type V | 95% | 11, 75% | 98% | 17, 91% | 98% | 23, 65% |
| Type VI | 95% | 12, 85% | 97% | 18, 91% | 97% | 24, 88% |

General Procedure for Preparation of Amine II

The octenyl glycoside I (1 eq.) and cysteamine hydrochloride (6 eq.) was taken up in dry CH₃OH (0.5 mL). The solution was degassed, placed under an argon atmosphere and irradiated (30 min). The solution was concentrated and subjected to C18 flash chromatography (1% CH₃COOH_{(aq)}→3:7 1% CH₃COOH_{(aq)}:CH₃OH). Concentration afforded the amine II as a colourless glass.

General Procedure for Preparation of Half Ester III

A solution of the amine II (1 eq.) in CH₃OH (20 mL) was neutralized with Amberlite IRA-400 (OH), filtered, concentrated and dried under vacuum. A solution of the amine in N,N-dimethylacetamide (2 mL) was treated with the homobifunctional adipic acid di-p-nitrophenyl ester linker (5 eq.) (Wu, X.; Ling, C.-C.; Bundle, D. R., Org. Lett., 2004, 6:4407-4410) and stirred (rt, 4 hours). The solution was then concentrated and subjected to C₁₈ flash chromatography (1% CH₃COOH_{(aq)}→1:1 1% CH₃COOH_{(aq)}:CH₃OH) to afford the half ester III as an unstable pale yellow coloured solid.

A Type I Half Ester 7

¹H NMR (500 MHz, CD₃OD): δ_{H} 8.32-8.26 (2H, m, Ph), 7.41-7.34 (2H, m, Ph), 5.23 (1H, d, J_{1″,2″} 3.8 Hz, H1″), 5.15 (1H, d, J_{1′″,2′″} 3.8 Hz, H1′″), 4.57 (1H, d, J_{1′,2′} 7.2, H1′), 4.36 (1H, q, J_{5″,6″} 6.7, H5″), 4.29 (1H, dd, J_{2′″,3′″} 10.6 Hz, J_{1′″,2′″} 3.8 Hz, H2′″), 4.25-4.17 (2H, m, H5′″, H1), 4.09 (1H, d, J_{3′,4′} 2.7 Hz, H4′), 3.95-3.62, 3.52-3.31 (23H, 2×m, H2″, H2′, H2, H3′″, H3″, H3′, H3, H4′″, H4″, H4, H5′″, H5′, H5, H6′″, H6′, H6, CH₂O, CH₂NH), 2.68-2.64 (2H, m, CH₂COO), 2.63-2.59 (2H, m, CH₂S), 2.55-2.50 (2H, m, CH₂S), 2.27-2.22 (2H, m, NHCOCH₂), 1.97 (6H, s, CH₃CO), 1.82-1.69 (4H, m, COCH₂(CH₂)₂CH₂COO), 1.59-1.48 (4H, m, OCH₂(CH₂)₆CH₂S), 1.41-1.24 (8H, m, OCH₂(CH₂)₆CH₂S), 1.20 (3H, d, J_{5″,6″} 6.7 Hz, H6″). ESIMS: m/z calcd [C₅₀H₈₀N₄O₂₅S]Na⁺: 1191.4725. Found: 1191.4720.

A Type II Half Ester 8

¹H NMR (500 MHz, CD₃OD): δ_{H} 8.32-8.26 (2H, m, Ph), 7.41-7.34 (2H, m, Ph), 5.33 (1H, d, J_{1″,2″} 3.8 Hz, H1′), 5.14 (1H, d, J_{1′″,2′″} 3.8 Hz, H1′″), 4.52 (1H, d, J_{1′,2′} 7.6 Hz, H1′), 4.38 (1H, d, J_{1,2} 8.3 Hz, H1), 4.34-4.29 (2H, m, H2′″, H5″), 4.16 (1H, dd, J_{5′″,6′″} 7.1 Hz, J_{5′″,6′″} 4.6 Hz, H5′″), 4.10 (1H, d, J_{3′,4′} 2.7 Hz, H4′), 3.99 (1H, dd, J_{2′,3′} 9.8 Hz, J_{1′,2′} 7.6 Hz, H2′), 3.93-3.62 (17H, m, H2″, H2, H3′″, H3″, H3′, H3, H4′″, H4″, H5′″, H6′″, H6′, H6, CH₂O), 3.54-3.50 (1H, m, CH₂NH), 3.47-3.41 (1H, m, CH₂O), 3.37-3.33, 3.31-3.26 (3H, m, H5, H5′, CH₂NH), 2.67-2.64 (2H, m, CH₂COO), 2.64-2.59 (2H, m, CH₂S), 2.55-2.50 (2H, m, CH₂S), 2.27-2.22 (2H, m, NHCOCH₂), 2.00 (3H, s, CH₃CO), 1.96 (3H, s, CH₃CO), 1.80-1.70 (4H, m, COCH₂(CH₂)₂CH₂COO), 1.60-1.48 (4H, m, OCH₂(CH₂)₆CH₂S), 1.40-1.25 (8H, m, OCH₂(CH₂)₆CH₂S), 1.21 (3H, d, J_{5″,6″} 6.6 Hz, H6″). ESIMS: m/z calcd [C₅₀H₈₀N₄O₂₅S]Na⁺: 1191.4725. Found: 1191.4715.

A Type III Half Ester 9

¹H NMR (500 MHz, CD₃OD): δ_{H} 8.30-8.26 (2H, m, Ph), 8.17 (1H, d, NH), 7.38-7.34 (2H, m, Ph), 5.19 (1H, d, J_{1″,2″} 4.0 Hz, H1″), 5.14 (1H, d, J_{1′″,2′″} 4.0 Hz, H1′″), 4.88 (1H, d, J_{1,2} 3.6 Hz, H1), 4.64 (1H, d, J_{1′,2′} 7.1 Hz, H1′), 4.32-4.22 (4H, m, H2, H2′″, H5″, H5′″), 4.19 (1H, d, J_{3,4} 2.5 Hz, H4), 4.08 (1H, d, J_{3′,4′} 2.6 Hz, H4′), 3.96 (1H, dd, J_{2,3} 11.2 Hz, J_{3,4} 2.5 Hz, H3), 3.93-3.81, 3.78-3.60 (15H, 2×m, H2′, H2″, H3′, H3″, H3′″, H4″, H4′″, H5, H6, H6′, H6′″, CH₂O), 3.49-3.46 (1H, m, H5′), 3.37-3.28 (3H, m, CH₂NH, CH₂O), 2.67-2.64 (2H, m, CH₂COO), 2.63-2.59 (2H, m, CH₂S), 2.54-2.50 (2H, m, CH₂S), 2.27-2.22 (2H, m, NHCOCH₂), 1.99 (3H, s, CH₃CO), 1.98 (3H, s, CH₃CO), 1.78-1.69 (4H, m, COCH₂(CH₂)₂CH₂COO), 1.59-1.51 (4H, m, OCH₂(CH₂)₆CH₂S), 1.42-1.26 (8H, m, OCH₂(CH₂)₆CH₂S), 1.23 (3H, d, J_{5″,6″} 6.5 Hz, H6″). ESIMS: m/z calcd [C₅₀H₈₀N₄O₂₅S]Na⁺: 1191.4725. Found: 1191.4717.

A Type IV Half Ester 10

¹H NMR (500 MHz, CD₃OD): δ_{H} 8.30-8.26 (2H, m, Ph), 7.40-7.35 (2H, m, Ph), 5.26 (1H, d, J_{1″,2″} 4.0 Hz, H1″), 5.15 (1H, d, J_{1′″,2′″} 3.7 Hz, H1′″), 4.56 (1H, d, J_{1′,2′} 7.5 Hz, H1′), 4.36-4.27 (2H, m, H2′″, H5″), 4.22-4.16 (2H, m, H1, H5′″), 4.13-4.04 (3H, m, H2, H4, H4′), 3.97 (1H, dd, J_{2′,3′} 9.7 Hz, J_{1′,2′} 7.5 Hz, H2′), 3.90-3.81, 3.79-3.66 (13H, 2×m, H2″, H3, H3′, H3′″, H4″, H4′″, H6, H6′, H6′″, CH₂O), 3.59 (1H, dd, J_{2″,3″} 10.2 Hz, J_{3″,4″} 3.1 Hz, H3″), 3.50 (1H, dd, J_{5′,6′} 6.0 Hz, J_{5′,6′} 6.0 Hz, H5′), 3.47-3.39 (2H, m, H5, CH₂O), 3.38-3.32 (2H, m, CH₂NH), 2.69-2.64 (2H, m, CH₂COO), 2.64-2.59 (2H, m, CH₂S), 2.55-2.50 (2H, m, CH₂S), 2.28-2.23 (2H, m, NHCOCH₂), 1.99 (6H, s, CH₃CO), 1.79-1.69 (4H, m, COCH₂(CH₂)₂CH₂COO), 1.59-1.47 (4H, m, OCH₂(CH₂)₆CH₂S), 1.41-1.26 (8H, m, OCH₂(CH₂)₆CH₂S), 1.22 (3H, d, J_{5″,6″} 6.5 Hz, H6″). ESIMS: m/z calcd [C₅₀H₈₀N₄O₂₅S]Na⁺: 1191.4725. Found: 1191.4715.

A Type V Half Ester 11

¹H NMR (500 MHz, CD₃OD): δ_{H} 8.31-8.26 (2H, m, Ph), 7.39-7.35 (2H, m, Ph), 5.29 (1H, d, J_{1″,2″} 3.9 Hz, H1′), 5.15 (1H, d, J_{1′″,2′″} 3.7 Hz, H1′″), 4.67-4.61 (2H, m, H1′, H5″), 4.32 (1H, dd, J_{2′″,3′″} 10.9 Hz, J_{1′″,2′″} 3.9 Hz, H2′″), 4.23-4.20 (1H, m, H1), 4.17 (1H, dd, J_{5′″,6′″} 6.9 Hz, J_{5′″,6′″} 4.9 Hz, H5′″), 4.13-4.06 (2H, m, H4, H4′), 4.01 (1H, dd, J_{2′,3′} 9.8 Hz, J_{1′,2′} 7.6 Hz, H2′), 3.93-3.79, 3.78-3.65, 3.62-3.46 (18H, 3×m, H2, H2″, H3, H3′, H3″, H3′″, H4″, H4′″, H5, H6, H6′, H6′″, CH₂O), 3.38-3.32 (2H, m, CH₂NH), 2.69-2.64 (2H, m, CH₂COO), 2.64-2.59 (2H, m, CH₂S), 2.56-2.50 (2H, m, CH₂S), 2.28-2.22 (2H, m, NHCOCH₂), 2.00 (3H, s, CH₃CO), 1.79-1.69 (4H, m, COCH₂(CH₂)₂CH₂COO), 1.65-1.51 (4H, m, OCH₂(CH₂)₆CH₂S), 1.41-1.26 (8H, m, OCH₂(CH₂)₆CH₂S), 1.21 (3H, d, J_{5″,6″} 6.5 Hz, H6″). ESIMS: m/z calcd [C₄₈H₇₇N₃O₂₅S]Na⁺: 1150.4459. Found: 1150.4456.

A Type VI Half Ester 12

¹H NMR (500 MHz, CD₃OD): δ_{H} 8.32-8.26 (2H, m, Ph), 7.40-7.34 (2H, m, Ph), 5.34 (1H, d, J_{1″,2″} 3.9 Hz, H1″), 5.15 (1H, d, J_{1′″,2′″} 3.7 Hz, H1′″), 4.51 (1H, d, J_{1′,2′} 7.6 Hz, H1′), 4.34-4.28 (2H, m, H2′″, H5″), 4.26 (1H, d, J_{1,2} 7.8 Hz, H1), 4.16 (1H, dd, J_{5′″,6′″} 7.0 Hz, J_{5′″,6′″} 4.6 Hz, H5′″), 4.10 (1H, d, J_{3′,4′} 2.6 Hz, H4′), 4.00 (1H, dd, J_{2′,3′} 9.8 Hz, J_{1′,2′} 7.6 Hz, H2′), 3.93-3.61 (15H, m, H2, H2″, H3′, H3′, H3′″, H4, H4″, H4′″, H6, H6′, H6′″, CH₂O), 3.56-3.50 (2H, m, H5′, CH₂O), 3.45 (1H, dd, J_{2,3} 9.1 Hz, J_{3,4} 9.1 Hz, H3), 3.37-3.21 (3H, m, H5, CH₂NH), 2.69-2.64 (2H, m, CH₂COO), 2.64-2.58 (2H, m, CH₂S), 2.55-2.50 (2H, m, CH₂S), 2.27-2.23 (2H, m, NHCOCH₂), 2.00 (3H, s, CH₃CO), 1.80-1.69 (4H, m, COCH₂(CH₂)₂CH₂COO), 1.64-1.52 (4H, m, OCH₂(CH₂)₆CH₂S), 1.42-1.25 (8H, m, OCH₂(CH₂)₆CH₂S), 1.21 (3H, d, J_{5″,6″} 6.6 Hz, H6″). ESIMS: m/z calcd [C₄₈H₇₇N₃O₂₅S]Na⁺: 1150.4459. Found: 1150.4455.

B Type I Half Ester 13

¹H NMR (500 MHz, CD₃OD): δ_{H} 8.30-8.25 (2H, m, Ph), 7.41-7.34 (2H, m, Ph), 5.20 (1H, d, J_{1″,2″} 3.9 Hz, H1″), 5.12 (1H, d, J_{1′″,2′″} 3.3 Hz, H1′″), 4.59-4.54 (1H, m, H1′), 4.34 (1H, q, J_{5″,6″} 6.5 Hz, H5″), 4.23-4.20 (1H, m, H1), 4.15 (1H, dd, J_{5′″,6′″} 6.6 Hz, J_{5′″,6′″} 4.8 Hz, H5′″), 4.09 (1H, s, H4′), 3.92-3.62 (17H, H2, H2′, H2″, H2′″, H3, H3′, H3″, H3′″, H4″, H4′″, H6, H6′, H6′″, CH₂O), 3.53 (1H, dd, J_{5′,6′} 7.6 Hz, J_{5′,6′} 4.4 Hz, H5′), 3.44-3.27 (5H, m, H4, H5, CH₂NH, CH₂O), 2.66-2.63 (2H, m, CH₂COO), 2.63-2.58 (2H, m, CH₂S), 2.55-2.49 (2H, m, CH₂S), 2.27-2.22 (2H, m, NHCOCH₂), 1.97 (3H, s, CH₃CO), 1.80-1.69 (4H, m, COCH₂(CH₂)₂CH₂COO), 1.58-1.43 (4H, m, OCH₂(CH₂)₆CH₂S), 1.40-1.24 (8H, m, OCH₂(CH₂)₆CH₂S), 1.17 (3H, d, J_{5″,6″} 6.5 Hz, H6″). ESIMS: m/z calcd [C₄₈H₇₇N₃O₂₅S]Na⁺: 1150.4459. Found: 1150.4455.

B Type II Half Ester 14

¹H NMR (500 MHz, CD₃OD): δ_{H} 8.33-8.27 (2H, m, Ph), 7.41-7.36 (2H, m, Ph), 5.34 (1H, d, J_{1″,2″} 3.9 Hz, H1″), 5.16 (1H, d, J_{1′″,2′″} 3.7 Hz, H1′″), 4.54 (1H, d, J_{1′,2′} 7.3 Hz, H1′), 4.39 (1H, d, J_{1,2} 8.4 Hz, H1), 4.30 (1H, q, J_{5″,6″} 6.6 Hz, H5″), 4.15-4.11 (2H, m, H4′, H5′″), 4.01-3.65 (17H, m, H2, H2′, H2″, H2′″, H3′, H3″, H3′″, H4, H4″, H4′″, H6, H6′, H6″, CH₂O), 3.63-3.55 (2H, m, H3, H5′), 3.48-3.42 (1H, m, CH₂O), 3.38-3.34 (2H, m, CH₂NH)), 3.33-3.27 (1H, m, H5), 2.70-2.65 (2H, m, CH₂COO), 2.65-2.60 (2H, m, CH₂S), 2.56-2.51 (2H, m, CH₂S), 2.30-2.24 (2H, m, NHCOCH₂), 1.97 (3H, s, CH₃CO), 1.81-1.71 (4H, m, COCH₂(CH₂)₂CH₂COO), 1.60-1.50 (4H, m, OCH₂(CH₂)₆CH₂S), 1.43-1.27 (8H, m, OCH₂(CH₂)₆CH₂S), 1.21 (3H, d, J_{5″,6″} 6.6 Hz, H6″). ESIMS: m/z calcd [C₄₈H₇₇N₃O₂₅S]Na⁺: 1150.4459. Found: 1150.4453.

B Type III Half Ester 15

¹H NMR (500 MHz, CD₃OD): δ_{H} 8.31-8.26 (2H, m, Ph), 8.18 (1H, m, J 7.8 Hz, NH) 7.39-7.35 (2H, m, Ph), 5.21-5.19 (1H, m, H1″), 5.14-5.13 (1H, m, H1′″), 4.89 (1H, d, J_{1,2} 3.5 Hz, H1), 4.65 (1H, d, J_{1′,2′} 6.9 Hz, H1′), 4.32-4.17 (4H, m, H2, H4, H5″, H5′″), 4.12 (1H, s, H4′), 3.98-3.60 (17H, m, H2′, H2″, H2′″, H3, H3′, H3″, H3′″, H4″, H4′″, H5, H6, H6′, H6′″, CH₂O), 3.53 (1H, dd, J_{5′,6′} 6.0 Hz, J_{5′,6′} 6.0 Hz, H5′), 3.38-3.30 (3H, m, CH₂O, CH₂NH), 2.68-2.64 (2H, m, CH₂COO), 2.63-2.59 (2H, m, CH₂S), 2.55-2.50 (2H, m, CH₂S), 2.29-2.22 (2H, m, NHCOCH₂), 1.99 (3H, s, CH₃CO), 1.79-1.69 (4H, m, COCH₂(CH₂)₂CH₂COO), 1.61-1.51 (4H, m, OCH₂(CH₂)₆CH₂S), 1.43-1.28 (8H, m, OCH₂(CH₂)₆CH₂S), 1.22 (3H, d, J_{5″,6″} 6.5 Hz, H6″). ESIMS: m/z calcd [C₄₈H₇₇N₃O₂₅S]Na⁺: 1150.4459. Found: 1150.4452.

B Type IV Half Ester 16

¹H NMR (500 MHz, CD₃OD): δ_{H} 8.31-8.26 (2H, m, Ph), 7.39-7.35 (2H, m, Ph), 5.25 (1H, d, J_{1″,2″} 4.1 Hz, H1″), 5.14 (1H, d, J_{1′″,2′″} 3.5 Hz, H1′″), 4.55 (1H, d, J_{1′,2′} 7.6 Hz, H1′), 4.30 (1H, q, J_{5″,6″} 6.5 Hz, H5″), 4.21-4.14 (2H, m, H1, H5′″), 4.13-4.05 (3H, m, H2, H4, H4′), 3.98 (1H, dd, J_{2′,3′} 9.5 Hz, J_{1′,2′} 7.6 Hz, H2′), 3.93-3.66 (15H, m, H2″, H2′″, H3, H3′, H3′″, H4″, H4′″, H5′″, H6, H6′, H6′″, CH₂O), 3.59 (1H, dd, J_{2″,3″} 10.2 Hz, J_{3″,4″} 3.0 Hz, H3″), 3.53-3.48 (2H, m, H5, H5′), 3.44-3.39 (1H, m, CH₂O), 3.38-3.33 (2H, m, CH₂NH), 2.68-2.64 (2H, m, CH₂COO), 2.64-2.59 (2H, m, CH₂S), 2.55-2.51 (2H, m, CH₂S), 2.27-2.23 (2H, m, NHCOCH₂), 1.96 (3H, s, CH₃CO), 1.80-1.69 (4H, m, COCH₂(CH₂)₂CH₂COO), 1.59-1.46 (4H, m, OCH₂(CH₂)₆CH₂S), 1.41-1.25 (8H, m, OCH₂(CH₂)₆CH₂S), 1.21 (3H, d, J_{5′,6″} 6.5 Hz, H6″). ESIMS: m/z calcd [C₄₈H₇₇N₃O₂₅S]Na⁺: 1150.4459. Found: 1150.4455.

B Type V Half Ester 17

¹H NMR (500 MHz, CD₃OD): δ_{H} 8.31-8.25 (2H, m, Ph), 7.39-7.33 (2H, m, Ph), 5.28 (1H, d, J_{1″,2′} 3.8 Hz, H1″), 5.14 (1H, d, J_{1′″,2′″} 3.5 Hz, H1′″), 4.65 (1H, d, J_{1′,2′} 7.5 Hz, H1′), 4.60 (1H, q, J_{5″,6″} 6.5 Hz, H5″), 4.23-4.19 (1H, m, H1), 4.17-4.14 (1H, m, H5′″), 4.14-4.09 (2H, m, H4, H4′), 4.00 (1H, dd, J_{2′,3′} 9.5 Hz, J_{1′,2′} 7.5 Hz, H2′), 3.94-3.66 (20H, 2×m, H2, H2′ H2′″, H3, H3′, H3″, H3′″, H4″, H4′″, H5, H5′, H5′″, H6, H6′, H6′″, CH₂O), 3.37-3.32 (2H, m, CH₂NH), 2.68-2.63 (2H, m, CH₂COO), 2.63-2.57 (2H, m, CH₂S), 2.54-2.49 (2H, m, CH₂S), 2.28-2.22 (2H, m, NHCOCH₂), 1.80-1.68 (4H, m, COCH₂(CH₂)₂CH₂COO), 1.64-1.49 (4H, m, OCH₂(CH₂)₆CH₂S), 1.41-1.26 (8H, m, OCH₂(CH₂)₆CH₂S), 1.19 (3H, d, J_{5″,6″} 6.5 Hz, H6″). ESIMS: m/z calcd [C₄₆H₇₄N₂O₂₅S]Na⁺: 1109.4194. Found: 1109.4188.

B Type VI Half Ester 18

¹H NMR (500 MHz, CD₃OD): δ_{H} 8.32-8.26 (2H, m, Ph), 7.40-7.35 (2H, m, Ph), 5.33 (1H, d, J_{1″,2″} 3.9 Hz, H1″), 5.15 (1H, d, J_{1′″,2′″} 3.7 Hz, H1′″), 4.52 (1H, d, J_{1′,2′} 7.5 Hz, H1′), 4.29 (1H, q, J_{5″,6″} 6.6 Hz, H5″), 4.26 (1H, d, J_{1,2} 7.8 Hz, H1), 4.14-4.09 (2H, m, H4′, H5′″), 3.99 (1H, dd, J_{2′,3′} 9.6 Hz, J_{1′,2′} 7.5 Hz, H2′), 3.94 (1H, dd, J_{2′,3′} 9.5 Hz, J_{3′,4′} 2.8 Hz, H3′), 3.92-2.62 (15H, m, H2″, H2′″, H3″, H3′″, H4, H4″, H4′″, H5, H6, H6′, H6′″, CH₂O), 3.58-3.50 (2H, m, H5′, CH₂O), 3.46 (1H, dd, J_{2,3} 9.1 Hz, J_{3,4} 9.1 Hz, H3), 3.38-3.32 (2H, m, CH₂NH), 3.30-3.22 (1H, m, H2), 2.69-2.64 (2H, m, CH₂COO), 2.64-2.59 (2H, m, CH₂S), 2.55-2.50 (2H, m, CH₂S), 2.27-2.22 (2H, m, NHCOCH₂), 1.80-1.70 (4H, m, COCH₂(CH₂)₂CH₂COO), 1.64-1.52 (4H, m, OCH₂(CH₂)₆CH₂S), 1.42-1.26 (8H, m, OCH₂(CH₂)₆CH₂S), 1.19 (3H, d, J_{5″,6″} 6.6 Hz, H6″). ESIMS: m/z calcd [C₄₆H₇₄N₂O₂₅S]Na⁺: 1109.4194. Found: 1109.4197.

H Type I Half Ester 19

¹H NMR (500 MHz, CD₃OD): δ_{H} 8.32-8.29 (2H, m, Ph), 7.40-7.34 (2H, m, Ph), 5.20 (1H, d, J_{1″,2″} 3.7 Hz, H1″), 4.54-4.52 (1H, m, H1′), 4.30 (1H, q, J_{5″,6″} 6.6 Hz, H5″), 4.27-4.24 (1H, m, H1), 3.91-3.63 (13H, m, H2, H2′, H2″, H3, H3′, H3″, H4′, H4″, H6, H6′, CH₂O), 3.52 (1H, dd, J_{5′,6′} 7.7 Hz, J_{5′,6′} 4.4 Hz, H5′), 3.45-3.37 (2H, m, H4, CH₂O), 3.37-3.33 (2H, m, CH₂NH), 3.32-3.28 (1H, m, H5), 2.68-2.64 (2H, m, CH₂COO), 2.64-2.59 (2H, m, CH₂S), 2.55-2.50 (2H, m, CH₂S), 2.28-2.23 (2H, m, NHCOCH₂), 1.97 (3H, s, CH₃CO), 1.80-1.70 (4H, m, COCH₂(CH₂)₂CH₂COO), 1.58-1.47 (4H, m, OCH₂(CH₂)₆CH₂S), 1.42-1.25 (8H, m, OCH₂(CH₂)₆CH₂S), 1.19 (3H, d, J_{5″,6″} 6.6 Hz, H6″). ESIMS: m/z calcd [C₄₂H₆₇N₃O₂₀S]Na⁺: 988.3931. Found: 988.3925.

H Type II Half Ester 20

¹H NMR (500 MHz, CD₃OD): δ_{H} 8.32-8.27 (2H, m, Ph), 7.39-7.34 (2H, m, Ph), 5.21 (1H, d, J_{1″,2″} 2.9 Hz, H1″), 4.50-4.47 (1H, m, H1′), 4.37 (1H, d, J_{1,2} 8.4 Hz, H1), 4.17 (1H, q, J_{5″,6″} 6.6 Hz, H5″), 3.91-3.52 (15H, m, H2, H2′, H2″, H3, H3′, H3″, H4, H4′, H4″, H5′, H6, H6′, CH₂O), 3.45-3.37 (1H, m, CH₂O), 3.37-3.31 (3H, m, H5, CH₂NH), 2.69-2.64 (2H, m, CH₂COO), 2.64-2.59 (2H, m, CH₂S), 2.56-2.50 (2H, m, CH₂S), 2.28-2.23 (2H, m, NHCOCH₂), 1.96 (3H, s, CH₃CO), 1.80-1.70 (4H, m, COCH₂(CH₂)₂CH₂COO), 1.60-1.48 (4H, m, OCH₂(CH₂)₆CH₂S), 1.41-1.25 (8H, m, OCH₂(CH₂)₆CH₂S), 1.20 (3H, d, J_{5″,6″} 6.6 Hz, H6″). ESIMS: m/z calcd [C₄₂H₆₇N₃O₂₀S]Na⁺: 988.3931. Found: 988.3926.

H Type III Half Ester 21

¹H NMR (500 MHz, CD₃OD): δ_{H} 8.30-8.25 (2H, m, Ph), 8.01 (1H, d, J 7.1 Hz, NH), 7.39-7.33 (2H, m, Ph), 5.11 (1H, d, J_{1″,2″} 3.8 Hz, H1″), 4.95 (1H, d, J_{1,2} 3.5 Hz, H1), 4.58 (1H, d, J_{1′,2′} 7.1 Hz, H1′), 4.28-4.14 (3H, m, H2, H4, H5″), 3.84-3.62 (12H, m, H2′, H2″, H3′, H3″, H4′, H4″, H5, H6, H6′, CH₂O), 3.53 (1H, dd, J_{5′,6′} 6.8 Hz, J_{5′,6′} 5.6 Hz, H5′), 3.37-3.31 (3H, m, CH₂O, CH₂NH), 2.68-2.63 (2H, m, CH₂COO), 2.63-2.59 (2H, m, CH₂S), 2.54-2.50 (2H, m, CH₂S), 2.27-2.22 (2H, m, NHCOCH₂), 1.97 (3H, s, CH₃CO), 1.79-1.68 (4H, m, COCH₂(CH₂)₂CH₂COO), 1.59-1.50 (4H, m, OCH₂(CH₂)₆CH₂S), 1.42-1.25 (11H, m, H6″, OCH₂(CH₂)₆CH₂S). ESIMS: m/z calcd [C₄₂H₆₇N₃O₂₀S]Na⁺: 988.3931. Found: 988.3927.

H Type IV Half Ester 22

¹H NMR (500 MHz, CD₃OD): δ_{H} 8.31-8.26 (2H, m, Ph), 7.49-7.34 (2H, m, Ph), 5.20 (1H, d, J_{1″,2″} 3.8 Hz, H1″), 4.53 (1H, d, J_{1′,2′} 7.2 Hz, H1′), 4.27-4.21 (2H, m, H1, H5″), 4.11-4.03 (2H, m, H2, H4), 3.89-3.63 (12H, m, H2′, H2″, H3, H3′, H3″, H4′, H4″, H6, H6′, CH₂O), 3.53-3.47 (2H, m, H5, H5′), 3.45-3.39 (1H, m, CH₂O), 3.38-3.32 (2H, m, CH₂NH), 2.69-2.64 (2H, m, CH₂COO), 2.64-2.59 (2H, m, CH₂S), 2.55-2.49 (2H, m, CH₂S), 2.28-2.22 (2H, m, NHCOCH₂), 1.96 (3H, s, CH₃CO), 1.79-1.69 (4H, m, COCH₂(CH₂)₂CH₂COO), 1.59-1.48 (4H, m, OCH₂(CH₂)₆CH₂S), 1.41-1.26 (8H, m, OCH₂(CH₂)₆CH₂S), 1.23 (3H, d, J_{5″,6″} 6.5 Hz, H6″). ESIMS: m/z calcd [C₄₂H₆₇N₃O₂₀S]Na⁺: 988.3931. Found: 988.3929.

H Type V Half Ester 23

¹H NMR (500 MHz, CD₃OD): δ_{H} 8.32-8.26 (2H, m, Ph), 7.39-7.34 (2H, m, Ph), 5.20 (1H, d, J_{1″,2″} 3.1 Hz, H1″), 4.63 (1H, d, J_{1′,2′} 7.2 Hz, H1′), 4.34 (1H, q, J_{5″,6″} 6.5 Hz, H5″), 4.37 (1H, d, J_{1,2} 7.5 Hz, H1), 4.10 (1H, d, J_{3,4} 2.4 Hz, H4), 3.91-3.47 (16H, m, H2, H2′, H2″, H3, H3′, H3″, H4′, H4″, H5, H5′, H6, H6′, CH₂O), 3.37-3.32 (2H, m, CH₂NH), 2.70-2.64 (2H, m, CH₂COO), 2.64-2.57 (2H, m, CH₂S), 2.55-2.50 (2H, m, CH₂S), 2.28-2.23 (2H, m, NHCOCH₂), 1.80-1.69 (4H, m, COCH₂(CH₂)₂CH₂COO), 1.65-1.52 (4H, m, OCH₂(CH₂)₆CH₂S), 1.42-1.26 (8H, m, OCH₂(CH₂)₆CH₂S), 1.23 (3H, d, J_{5″,6″} 6.5 Hz, H6″). ESIMS: m/z calcd [C₄₀H₆₄N₂O₂₀S]Na⁺: 947.3665. Found: 947.3659.

H Type VI Half Ester 24

¹H NMR (500 MHz, CD₃OD): δ_{H} 8.30-8.25 (2H, m, Ph), 7.39-7.33 (2H, m, Ph), 5.22 (1H, d, J_{1″,2″} 3.2 Hz, H1″), 4.47 (1H, d, J_{1′,2′} 7.1 Hz, H1′), 4.25 (1H, d, J_{1,2} 7.8 Hz, H1), 4.16 (1H, q, J_{5″,6″} 6.6 Hz, H5″), 3.90-3.62 (12H, m, H2′, H2″, H3′, H3″, H4, H4′, H4″, H6, H6′, CH₂O), 3.56-3.49 (2H, m, H5′, CH₂O), 3.47 (1H, dd, J_{2,3} 9.2 Hz, J_{3,4} 9.1 Hz, H3), 3.36-3.30 (3H, m, H5, CH₂NH), 3.23 (1H, dd, J_{2,3} 9.2 Hz, J_{1,2} 7.8 Hz, H2), 2.69-2.63 (2H, m, CH₂COO), 2.63-2.58 (2H, m, CH₂S), 2.54-2.49 (2H, m, CH₂S), 2.27-2.21 (2H, m, NHCOCH₂), 1.79-1.69 (4H, m, COCH₂(CH₂)₂CH₂COO), 1.64-1.52 (4H, m, OCH₂(CH₂)₆CH₂S), 1.41-1.26 (8H, m, OCH₂(CH₂)₆CH₂S), 1.19 (3H, d, J_{5″,6″} 6.6 Hz, H6″). ESIMS: m/z calcd [C₄₀H₆₄N₂O₂₀S]Na⁺: 947.3665. Found: 947.3659.

General Procedure for Preparation of Bovine Serum Albumin (BSA) Conjugates of ABO Glycans:

BSA (2.5 mg) was dissolved in sodium phosphate buffer pH 7.5 (0.5 mL), and the PNP ester of a glycan (e.g., 7, Scheme 1) at a selected molar ratio was dissolved in dry DMF (25 μL). The resulting the solution was then injected into the reaction medium dropwise and the reaction was left rotating for 24 h at room temperature. The mixture was then dialysed in a 4 L beaker against five changes of deionized water, with each change lasting for at least 4 h. The solution was then lyphophilized to obtain a white solid. MALDI mass spectrometry was performed to determine the number of antigens on the BSA (Table 3)

TABLE 3

| | Antigen Loading on BSA as | Molar Ratio of |
|---|---|---|
| Product | determined by MALDI-MS | BSA:PNP Ester |
| A Type I-BSA | 12 | 1:40 |
| A Type II-BSA | 11 | 1:30 |
| A Type III-BSA | 11 | 1:40 |
| A Type IV-BSA | 9 | 1:40 |
| A Type V-BSA | 12 | 1:50 |
| A Type VI-BSA | 6 | 1:30 |
| B Type I-BSA | 9 | 1:40 |
| B Type II-BSA | 8 | 1:40 |
| B Type III-BSA | 11 | 1:40 |
| B Type IV-BSA | 12 | 1:40 |
| B Type V-BSA | 12 | 1:50 |
| B Type VI-BSA | 11 | 1:40 |
| H Type I-BSA | 18 | 1:40 |
| H Type II-BSA | 11 | 1:40 |
| H Type III-BSA | 16 | 1:40 |
| H Type IV-BSA | 12 | 1:40 |
| H Type V-BSA | 14 | 1:40 |
| H Type VI-BSA | 20 | 1:40 |

Conjugation of Antigens to Glycan Microarray Slides

Amine functionalized microarray slides were purchased from Arrayit (Superamine 2, SMM2). Prior to printing these slides were dipped in a solution of triethylamine (3%) in methanol. The slides were then washed with methanol and dried by a steady stream of argon gas. The preparation of the ABO histo-blood group antigen subtypes is described above. Approximately 1.7 mg of the antigen was taken up in DMA (120 μL) and the solution printed onto the array slides using a pin microarray device. Prior to utilizing the slides they were washed by dipping in deionized water followed by methanol.

N-hydroxysuccinimide functionalized microarray slides were purchased from Schott AG (Slide H). The preparation of the ABO histo-blood group antigen subtypes is described above. Each antigen was dissolved in 300 mM phosphate buffer, pH 8.5 containing 0.005% Tween-20 at a final antigen concentration of either 100 μM or 10 μM. Microarrays were printed by robotic pin deposition of ˜0.6 nL of each solution; each concentration of all antigens was printed in replicates of three. Printed slides were allowed to react in an atmosphere of 80% humidity for 30 min followed by desiccation overnight. Remaining N-hydroxysuccinimide groups were blocked by immersion in buffer (50 mM ethanolamine in 50 mM borate buffer, pH 9.2) for 1 h. Slides were rinsed with water, dried, and stored in desiccators at room temperature before use.

Epoxide functionalized microarray slides were purchased from Schott AG (Slide E). The preparation of the ABO histo-blood group antigen subtype BSA conjugates is described above. Arrays were prepared generally as described in Gildersleeve, et al., Curr. Protoc. Chem. Biol. 2010, 2, 37-53. Each BSA conjugate was dissolved in phosphate buffered saline, pH 7.4 containing 0.006% TritonX-100 and 2.5% glycerol at a final concentration of 125 μg/mL. Microarrays were printed by robotic pin deposition of ˜0.6 nL of each solution; each concentration of BSA conjugate was printed in replicates of three. The printed slides were allowed to react in an atmosphere of 80% humidity for 30 min followed by desiccation overnight. Slides were rinsed with water, dried, and stored desiccated at 20° C. before use.

Hybridization and Detection of Antibodies

Microarray slides (up to four slides per cassette) were loaded into the 96-well format hybridization cassette (AHC4×24, Arrayit Corporation) to allow multiple microarrays to be reacted and processed simultaneously. After washing with PBS/Tween-20 microarrays were blocked with 1-percent bovine serum albumin/PBS and/or 3-percent human serum albumin/PBS for one hour at room temperature. Diluted plasma (50 μL at 1:20-1:150 in blocking buffer) or serially diluted reference serum was reacted for 30 minutes at room temperature. After the washing with PBS/Tween20, bound antibodies were detected using fluorochrome (Dylight 549™ or Dylight 649™) conjugated goat anti-human IgM, IgG (including, IgG1, IgG2, IgG3 and IgG4) or IgA antibody (Jackson ImmunoResearch Laboratories, Inc.). Microarray slides were scanned immediately on the Nimblegen™ MS200 microarray scanner (Roche Nimblegen, Inc.) at 2-5 μm resolution. Scanned images were analyzed using Imagene™ software (Biodiscovery, Inc.) and concentration of antigen-specific plasma or serum antibodies are derived from the standard curve generated with reference serum.

### Example 1

### Evaluation of a Blood Group-0 Individual

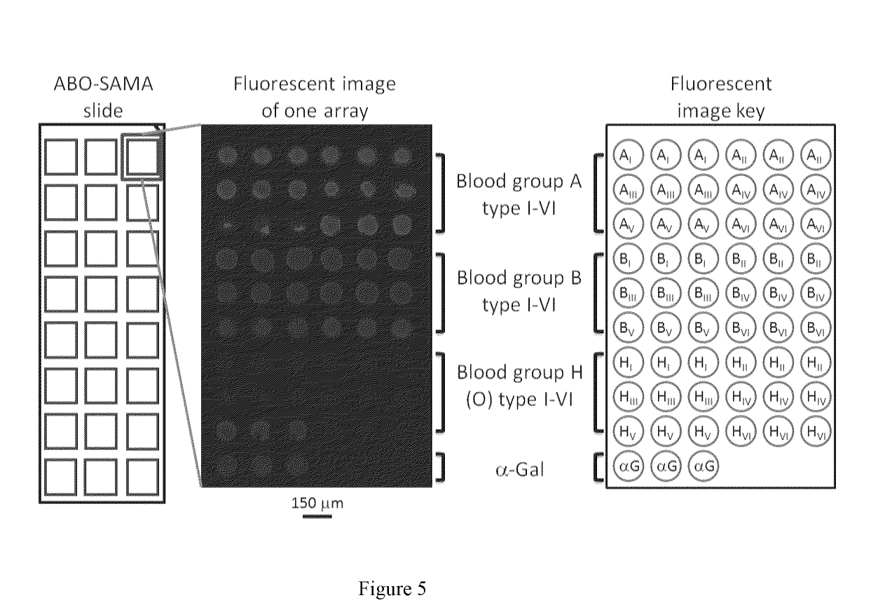

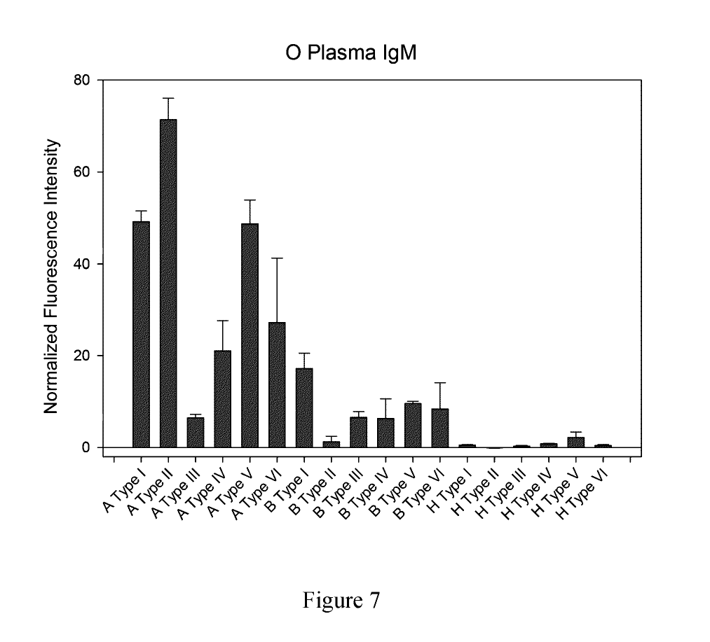

Plasma from a blood group-0 individual was analyzed using the glycan array and bound antibodies were detected using anti-human IgM-DyLight649. The array used in this example included an α-Gal positive control as shown in the schematic in FIG. 5. As seen in the fluorescent image in FIG. 7, this blood group-0 individual produced antibodies specific for all 12 A- & B-subtypes as well as H type V (absent in human tissues), but lacked antibodies to all other H-subtypes. As noted above, α-Gal is a non-human antigen similar to the ABO antigens and the signal corresponding to the location of the α-Gal antigen shown here indicated the reaction of anti-α-Gal antibodies in the plasma sample from the individual with the immobilized α-Gal antigen on the array.

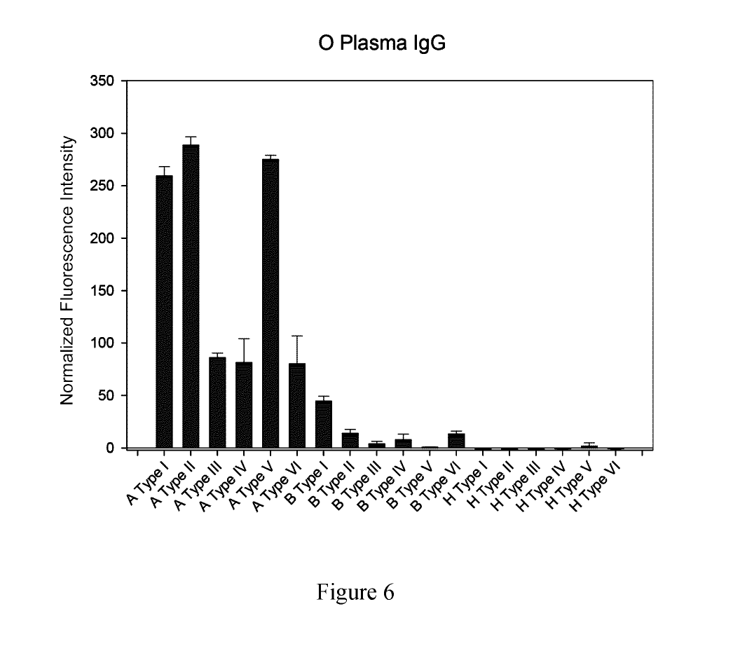

Plasma from a blood group-0 individual was analyzed using the glycan array for both IgG (FIG. 6) and IgM (FIG. 7) isotype antibodies towards the ABO subtypes. All blood group-O individuals would be expected to produce ‘natural’ antibodies against all six subtypes of both A and B antigens. In this patient, very high IgG isotype antibody levels towards the A type I, II and V antigens were observed while lower but still significant levels of antibodies towards the A type III, IV and VI were measured. Antibodies towards the B type I antigen were moderate, whilst for the remaining B subtypes the antibody levels were quite low. As expected IgG isotype antibodies against the H antigens were not detectable, except H type V (absent in human tissues). A similar trend was observed with the IgM isotype antibodies.

These results demonstrate the ability of the array to reliably differentiate amongst ABO antibody isotypes, indicating that the device is robust and can provide detailed clinical information.

### Example 2

### Pre- and Post-Transplant Monitoring of Antibodies Using Glycan Array

ABO subtype I-VI antigens are differentially expressed in tissues and organs in a secretor-dependent or independent manner. In the setting of ABO-incompatible organ transplantation, assessment of antibodies against each subtype is crucial for a given organ. In the heart, for example, only type II ABH antigen structures are expressed in vascular endothelia and it would be important to measure antibodies against type II antigens (donor-specific antibodies) following ABO-incompatible heart transplantation.

The glycan array is a useful tool for ascertaining reliably the presence/absence of donor-specific antibodies after ABO-incompatible transplantation. Until now, the traditional agglutination assay was the only means to follow ABO antibody titres in patients in the months and years following ABO-incompatible transplantation. Without the knowledge of which ABO subtype antibodies are actually present, an unexpectedly high titre against the donor blood group as detected by the standard red cell agglutination test would result in potentially unnecessary and expensive interventions to remove those antibodies, which may be irrelevant, ie not specific to antigens expressed in the transplanted organ.

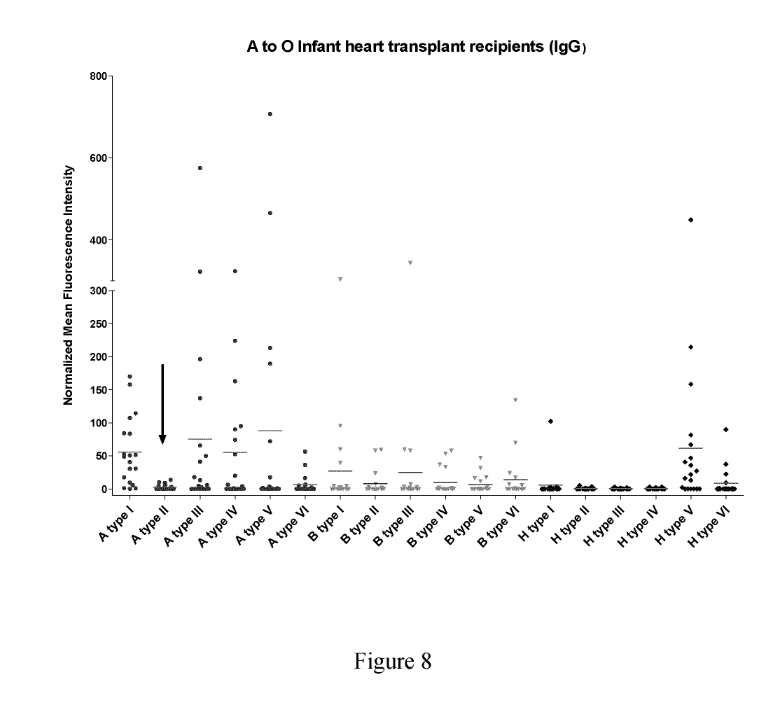

Using the glycan array, sera were analyzed from 20 blood group-O patients who had received heart transplants as infants from blood group-A donors. The results (FIG. 8) clearly demonstrate the specific absence of anti-A IgG antibodies (FIG. 8, arrow) against the A-antigen subtype (type II) expressed in cardiac tissue (ie, the transplanted organ), despite the presence of other non-damaging antibodies against the donor blood group. Use of the glycan array for pre- and post-transplant monitoring gives precise delineation of which ABO subtype-specific antibodies are present in the patient's blood, and will therefore diminish the use of unnecessary and costly interventions to remove antibodies that are not actually harmful to the transplanted organ. In the presence of ABO antibodies, frequent antibody depletion strategies are often required pre- and/or post-transplantation. The glycan array can also be a valuable tool in the monitoring of donor specific antibodies following interventions and their effectiveness.

### Example 3

### Use of Glycan Array in ABO-Compatible Transplant and Transfusion

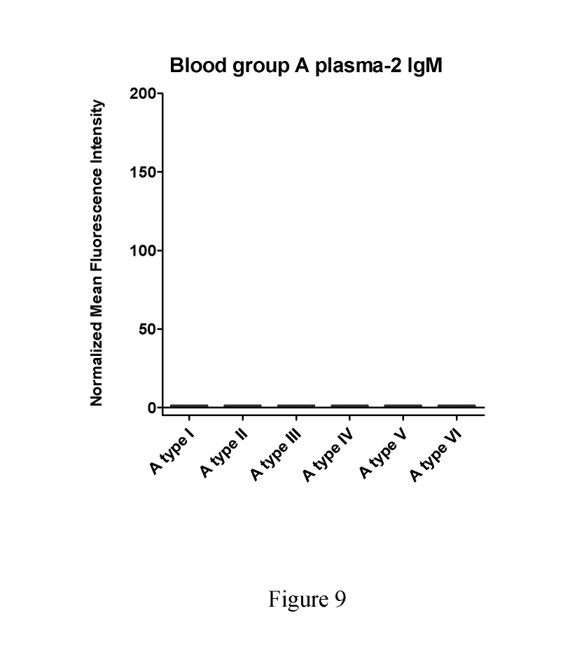

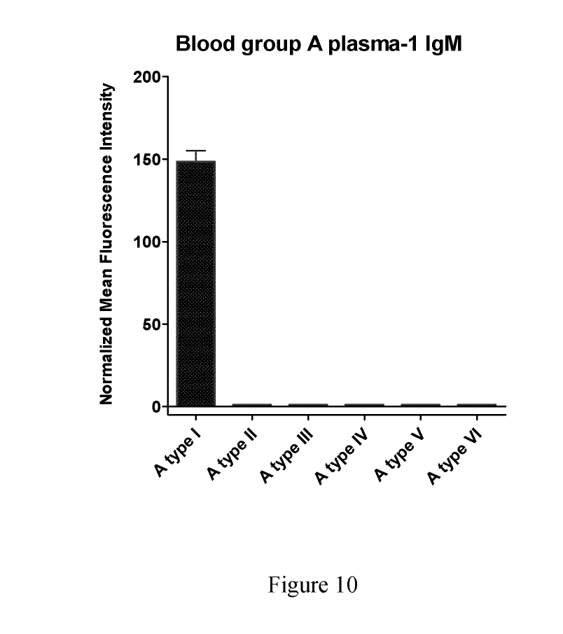

Blood group-A individuals generally develop natural antibodies against B antigens that are not expressed in their cells and tissues but not against self-antigens (A) that are widely expressed in cells and tissues throughout the body (FIG. 9). A type I antigens, however, are expressed only in lining epithelia and glandular epithelia in a secretor-dependent manner (Fujitani, N., et al., Glycoconj. 12000, 17(5): 331-338; and Fujitani, N., et al. J. Histochem. Cytochem. 2000, 48(12): 1649-1656). Although erythrocytes generally do not synthesize type I ABH structures, their expression in secretors is due to absorption of circulating glycolipids from plasma (Clausen, H. and S. Hakomori Vox Sang 1989, 56(1): p. 1-20). Therefore, in this manner some individuals who are non-secretors may develop serum antibodies against A type I antigens not expressed in their cells and tissues (FIG. 10). Transfusion related complications may arise when these individuals receive red cell transfusions from blood group-A secretor blood donors. Similarly, acute or chronic antibody mediated rejection may occur when these individuals receive organ transplant from blood group-A secretor donors (considered to be ABO-compatible). Thus, the glycan array can be a valuable tool in dealing with transfusion related complications and improving transplant outcomes.

### Example 4

### Determination of Subgroup Using the Glycan Array

A small population of blood group A₂ individuals produce antibodies against A types III and IV, because they do not express these subtypes. In contrast, blood group A₁ individuals do not produce antibodies against any of the A subtype antigens, except non-secretors as shown in example 3. The glycan array can reliably detect the antibodies to these antigens and could therefore theoretically be used to identify blood group A₂ versus A₁ individuals.

These examples, results of testing plasma or serum isolated directly from healthy volunteers and patients, are indicative of the wealth of knowledge that can be obtained using the ABO glycan array. The application of this knowledge toward predicting transplant success and monitoring the results of therapeutic interventions is invaluable to a physician.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method of identifying the ABO histo-blood subtype antibody profile of a subject comprising:
(a) identifying the presence or absence of antibodies specific for at least one ABO histo-blood group antigen subtype in a biological sample from said subject; and
(b) using the information from step (a) to generate the ABO antigen subtype antibody profile of the subject,
wherein step (a) optionally includes quantifying the amount of the at least one anti-ABO histo-blood group antigen subtype antibody in the biological sample.

2. The method of claim 1, wherein step (a) comprises identifying the presence or absence of antibodies specific for up to 18 ABO histo-blood group antigen subtypes in a biological sample from said subject.

3. The method of claim 1 or 2, wherein the step of identifying the presence or absence of antibodies specific for at least one ABO histo-blood group antigen subtype comprises:
(a) applying the biological sample from the subject to a glycan microarray or macroarray containing a plurality of immobilized ABO histo-blood group antigens;
(b) incubating the biological sample with the glycan microarray or macroarray such that binding complexes form between anti-ABO histo-blood group antigen subtype antibodies present in the sample and the bound antigens; and
(c) detecting the binding complexes and identifying corresponding antigens.

4. The method of claim 3, wherein the immobilized ABO antigen subtypes are selected from the group consisting of:
A type I α-D-GalpNAc-(1→3)-[α-L-Fucp-(1→2)]-β-D-Galp-(1→3)-β-D-GlcpNAc-R,
A type II α-D-GalpNAc-(1→3)-[α-L-Fucp-(1→2)]-β-D-Galp-(1→4)-β-D-GlcpNAc-R,
A type III α-D-GalpNAc-(1→3)-[α-L-Fucp-(1→2)]-β-D-Galp-(1→3)-α-D-GalpNAc-R,
A type IV α-D-GalpNAc-(1→3)-[α-L-Fucp-(1→2)]-β-D-Galp-(1→3)-β-D-GalpNAc-R,
A type V α-D-GalpNAc-(1→3)-[α-L-Fucp-(1→2)]-β-D-Galp-(1→3)-β-D-Galp-R,
A type VI α-D-GalpNAc-(1→3)-[α-L-Fucp-(1→2)]-β-D-Galp-(1→4)-β-D-Glcp-R,
B type I α-D-Galp-(1→3)-[α-L-Fucp-(1→2)]-β-D-Galp-(1→3)-β-D-GlcpNAc-R,
B type II α-D-Galp-(1→3)-[α-L-Fucp-(1→2)]-β-D-Galp-(1→4)-β-D-GlcpNAc-R,
B type III α-D-Galp-(1→3)-[α-L-Fucp-(1→2)]-β-D-Galp-(1→3)-α-D-GalpNAc-R,
B type IV α-D-Galp-(1→3)-[α-L-Fucp-(1→2)]-β-D-Galp-(1→3)-β-D-GalpNAc-R,
B type V α-D-Galp-(1→3)-[α-L-Fucp-(1→2)]-β-D-Galp-(1→3)-β-D-Galp-R,
B type VI α-D-Galp-(1→3)-[α-L-Fucp-(1→2)]-β-D-Galp-(1→4)-β-D-Glcp-R,
H type I α-L-Fucp-(1→2)-β-D-Galp-(1→3)-β-D-GlcpNAc-R,
H type II α-L-Fucp-(1→2)-β-D-Galp-(1→4)-β-D-GlcpNAc-R,
H type III α-L-Fucp-(1→2)-β-D-Galp-(1→3)-α-D-GalpNAc-R,
H type IV α-L-Fucp-(1→2)-β-D-Galp-(1→3)-β-D-GalpNAc-R,
H type V α-L-Fucp-(1→2)-β-D-Galp-(1→3)-β-D-Galp-R, and
H type VI α-L-Fucp-(1→2)-β-D-Galp-(1→4)-β-D-Glcp-R,
wherein R is an amine, a linker group for covalently attaching the antigen subtypes to the microarray or macroarray; or a protein, such as, bovine serum albumin (BSA), human serum albumin (HSA), keyhole limpet hemocyanin (KLH), chicken serum albumin (CSA), tetanus toxoid (TT) or diphtheria toxin mutant Cross-reactive material 197 Protein (CRM197).

5. The method of claim 3 or 4, wherein the step of detecting the binding complexes comprises:
(i) incubating the binding complexes immobilized on the glycan microarray or macroarray with a labeled secondary antibody that binds to the binding complexes and/or the bound anti-ABO histo-blood group antigen subtype antibodies; and
(ii) detecting the bound labeled secondary antibody.

6. The method of claim 5, wherein the secondary antibody is an anti-human antibody, which is anti-human IgG (IgG1, IgG2, IgG3, IgG4), IgM, IgE, IgD and/or IgA or any combination of these antibodies.

7. The method of claim 5 or 6, wherein the label on the secondary antibody can be detected separately or collectively.

8. The method of any one of claims 4-6, wherein the label comprises a fluorophore or a chromophore.

9. The method of any one of claims 3-8, which comprises qualitatively detecting the presence or absense of anti-ABO histo-blood group antigen subtype antibodies in the biological sample.

10. The method of any one of claims 3-8, which comprises quantitatively detecting the amount of anti-ABO histo-blood group antigen subtype antibodies in the biological sample.

11. The method of any one of claims 1-10, wherein the biological sample is plasma or serum.

12. The method of any one of claims 1-11, additionally comprising the step of comparing the ABO histo-blood antigen subtype antibody profile of the subject with an ABO histo-blood group, ABO histo-blood subgroup or ABO histo-blood antigen subtype profile of a donor tissue or blood product.

13. A method of evaluating the suitability of a donor tissue or blood product for a recipient subj ect comprising:
(a) determining an anti-ABO antigen subtype antibody profile using a biological sample from said recipient subject;
(b) determining the ABO histo-blood group or ABO histo-blood subgroup of the donor blood or tissue product; and
(c) determining the suitability of the donor tissue or blood product for donation to said recipient subject based on a comparison of the determined anti-ABO antigen subtype antibody profile of the recipient subject with the ABO histo-blood group or ABO histo-blood subgroup of the donor blood or tissue product.

14. The method of claim 13, wherein the step of determining the anti-ABO antigen subtype antibody profile comprises:
(a) applying the biological sample from the subject to a glycan microarray or macroarray containing a plurality of immobilized ABO antigen;
(b) incubating the biological sample with the glycan microarray or macroarray such that binding complexes form between anti-ABO histo-blood group antigen subtype antibodies present in the sample and the bound antigens; and
(c) detecting the binding complexes and identifying corresponding antigens.

15. The method of claim 13 or 14, wherein the step of determining the ABO histo-blood subgroup of the donor blood or tissue product comprises:
(a) applying a biological sample from the donor to a glycan microarray or macroarray containing a plurality of immobilized ABO antigen;
(b) incubating the biological sample with a second glycan microarray or macroarray such that binding complexes form between anti-ABO histo-blood group antigen subtype antibodies present in the donor's sample and the bound antigens;
(c) detecting the binding complexes and identifying corresponding antigens; and
(d) identifying the ABO histo-blood subgroup of the donor blood or tissue product based on the amount and/or quantity of anti-ABO histo-blood group antigen subtype antibodies present in said donor blood or tissue product.

16. The method of claim 14 or 15, wherein the immobilized ABO antigen subtypes are selected from the group consisting of:
A type I α-D-GalpNAc-(1→3)-[α-L-Fucp-(1→2)]-β-D-Galp-(1→3)-β-D-GlcpNAc-R,
A type II α-D-GalpNAc-(1→3)-[α-L-Fucp-(1→2)]-β-D-Galp-(1→4)-β-D-GlcpNAc-R,
A type III α-D-GalpNAc-(1→3)-[α-L-Fucp-(1→2)]-β-D-Galp-(1→3)-α-D-GalpNAc-R,
A type IV α-D-GalpNAc-(1→3)-[α-L-Fucp-(1→2)]-β-D-Galp-(1→3)-β-D-GalpNAc-R,
A type V α-D-GalpNAc-(1→3)-[α-L-Fucp-(1→2)]-β-D-Galp-(1→3)-β-D-Galp-R,
A type VI α-D-GalpNAc-(1→3)-[α-L-Fucp-(1→2)]-β-D-Galp-(1→4)-β-D-Glcp-R,
B type I α-D-Galp-(1→3)-[α-L-Fucp-(1→2)]-β-D-Galp-(1→3)-β-D-GlcpNAc-R,
B type II α-D-Galp-(1→3)-[α-L-Fucp-(1→2)]-β-D-Galp-(1→4)-β-D-GlcpNAc-R,
B type III α-D-Galp-(1→3)-[α-L-Fucp-(1→2)]-β-D-Galp-(1→3)-α-D-GalpNAc-R,
B type IV α-D-Galp-(1→3)-[α-L-Fucp-(1→2)]-β-D-Galp-(1→3)-β-D-GalpNAc-R,
B type V α-D-Galp-(1→3)-[α-L-Fucp-(1→2)]-β-D-Galp-(1→3)-β-D-Galp-R,
B type VI α-D-Galp-(1→3)-[α-L-Fucp-(1→2)]-β-D-Galp-(1→4)-β-D-Glcp-R,
H type I α-L-Fucp-(1→2)-β-D-Galp-(1→3)-β-D-GlcpNAc-R,
H type II α-L-Fucp-(1→2)-β-D-Galp-(1→4)-β-D-GlcpNAc-R,
H type III α-L-Fucp-(1→2)-β-D-Galp-(1→3)-α-D-GalpNAc-R,
H type IV α-L-Fucp-(1→2)-β-D-Galp-(1→3)-β-D-GalpNAc-R,
H type V α-L-Fucp-(1→2)-β-D-Galp-(1→3)-β-D-Galp-R, and
H type VI α-L-Fucp-(1→2)-β-D-Galp-(1→4)-β-D-Glcp-R,
wherein R is a linker group for covalently attaching the antigen subtypes to the microarray or macroarray.

17. The method of any one of claims 14-16, wherein the step of detecting the binding complexes comprises:
incubating the binding complexes immobilized on the glycan microarray or macroarray with a labeled secondary antibody that binds to the binding complexes and/or the bound anti-ABO histo-blood group antigen subtype antibodies; and
(ii) detecting the bound labeled secondary antibody.

18. The method of claim 17, wherein the secondary antibody is an anti-human antibody, which is anti-human IgG (IgG1, IgG2, IgG3, IgG4), IgM, IgE, IgD and/or IgA or any combination of these antibodies.

19. The method of claim 17 or 18, wherein the label on the secondary antibody can be detected separately or collectively.

20. The method of any one of claims 17-19, wherein the label comprises a fluorophore or a chromophore.

21. The method of any one of claims 14-20, which comprises qualitatively detecting the presence or absence of anti-ABO histo-blood group antigen subtype antibodies in the biological sample.

22. The method of any one of claims 14-20, which comprises quantitatively detecting the amount of anti-ABO histo-blood group antigen subtype antibodies in the biological sample.

23. The method of any one of claims 13-22, wherein the biological sample is plasma or serum.

24. The method of any one of claims 13-23, wherein the recipient subject is a transfusion recipient or a transplant recipient.

25. The method of any one of claims 13-24, which additionally comprises testing for compatibility using traditional techniques.

26. The method of any one of claims 13-25, wherein the recipient subject is monitored for adverse reaction following transfusion of the donor blood product or transplantation of the donor tissue product by using the glycan microarray or macroarray to screen for a change in the anti-ABO antigen subtype antibody profile as an indicator of an adverse reaction.

27. A method of monitoring a recipient subject for an adverse reaction following transfusion with a donor blood product or transplantation with a donor tissue, said method comprising:
(a) determining a first anti-ABO antigen subtype antibody profile using a first biological sample from said recipient subject;
(b) determining a second anti-ABO antigen subtype antibody profile using a second biological sample from said recipient subject, wherein the second;
(c) comparing the first anti-ABO antigen subtype antibody profile with the second anti-ABO antigen subtype antibody profile to identify a change in the anti-ABO antigen subtype antibody profile as an indicator of an adverse reaction; and, optionally,
(d) repeating steps (a) to (c).

28. The method of claim 27, wherein the step of determining each anti-ABO antigen subtype antibody profile comprises:
(a) applying the biological sample to a glycan microarray or macroarray containing a plurality of immobilized ABO antigens;
(b) incubating the biological sample with the glycan microarray or macroarray such that binding complexes form between anti-ABO histo-blood group antigen subtype antibodies present in the sample and the bound antigens; and
(c) detecting the binding complexes and identifying corresponding antigens.

29. The method of claim 28, wherein the immobilized ABO antigen subtypes are selected from the group consisting of:
A type I α-D-GalpNAc-(1→3)-[α-L-Fucp-(1→2)]-β-D-Galp-(1→3)-β-D-GlcpNAc-R,
A type II α-D-GalpNAc-(1→3)-[α-L-Fucp-(1→2)]-β-D-Galp-(1→4)-β-D-GlcpNAc-R,
A type III α-D-GalpNAc-(1→3)-[α-L-Fucp-(1→2)]-β-D-Galp-(1→3)-α-D-GalpNAc-R,
A type IV α-D-GalpNAc-(1→3)-[α-L-Fucp-(1→2)]-β-D-Galp-(1→3)-β-D-GalpNAc-R,
A type V α-D-GalpNAc-(1→3)-[α-L-Fucp-(1→2)]-β-D-Galp-(1→3)-β-D-Galp-R,
A type VI α-D-GalpNAc-(1→3)-[α-L-Fucp-(1→2)]-β-D-Galp-(1→4)-β-D-Glcp-R,
B type I α-D-Galp-(1→3)-[α-L-Fucp-(1→2)]-β-D-Galp-(1→3)-β-D-GlcpNAc-R,
B type II α-D-Galp-(1→3)-[α-L-Fucp-(1→2)]-β-D-Galp-(1→4)-β-D-GlcpNAc-R,
B type III α-D-Galp-(1→3)-[α-L-Fucp-(1→2)]-β-D-Galp-(1→3)-α-D-GalpNAc-R,
B type IV α-D-Galp-(1→3)-[α-L-Fucp-(1→2)]-β-D-Galp-(1→3)-β-D-GalpNAc-R,
B type V α-D-Galp-(1→3)-[α-L-Fucp-(1→2)]-β-D-Galp-(1→3)-β-D-Galp-R,
B type VI α-D-Galp-(1→3)-[α-L-Fucp-(1→2)]-β-D-Galp-(1→4)-β-D-Glcp-R,
H type I α-L-Fucp-(1→2)-β-D-Galp-(1→3)-β-D-GlcpNAc-R,
H type II α-L-Fucp-(1→2)-β-D-Galp-(1→4)-β-D-GlcpNAc-R,
H type III α-L-Fucp-(1→2)-β-D-Galp-(1→3)-α-D-GalpNAc-R,
H type IV α-L-Fucp-(1→2)-β-D-Galp-(1→3)-β-D-GalpNAc-R,
H type V α-L-Fucp-(1→2)-β-D-Galp-(1→3)-β-D-Galp-R, and
H type VI α-L-Fucp-(1→2)-β-D-Galp-(1→4)-β-D-Glcp-R,
wherein R is a linker group for covalently attaching the antigen subtypes to the microarray or macroarray.

30. The method of claim 28 or 29, wherein the step of detecting the binding complexes comprises:
incubating the binding complexes immobilized on the glycan microarray or macroarray with a labeled secondary antibody that binds to the binding complexes and/or the bound anti-ABO histo-blood group antigen subtype antibodies; and
(ii) detecting the bound labeled secondary antibody.

31. The method of claim 30, wherein the secondary antibody is an anti-human antibody, which is anti-human IgG (IgG1, IgG2, IgG3, IgG4), IgM, IgE, IgD and/or IgA or any combination of these antibodies.

32. The method of claim 30 or 31, wherein the label on the secondary antibody can be detected separately or collectively.

33. The method of any one of claims 30-32, wherein the label comprises a fluorophore or a chromophore.

34. The method of any one of claims 27-33, which comprises qualitatively detecting the presence or absence of anti-ABO histo-blood group antigen subtype antibodies in the biological sample.

35. The method of any one of claims 27-33, which comprises quantitatively detecting the amount of anti-ABO histo-blood group antigen subtype antibodies in the biological sample.

36. The method of any one of claims 27-35, wherein the biological sample is blood or serum.

37. The method of any one of claims 27-36, wherein the recipient subject is a transplant recipient or a transfusion recipient.

38. An assay kit for determining an anti-ABO antigen subtype antibody profile of a subject, said kit comprising:
(a) a glycan microarray or macroarray containing a plurality of immobilized ABO antigens; and
(b) instructions for use.

39. The kit of claim 38, additionally comprising an immobilized positive control antigen.

40. The kit of claim 39, wherein the positive control antigen is α-Gal.

41. The assay kit of any one of claims 38-40, wherein the immobilized ABO antigen subtypes are selected from the group consisting of:
A type I α-D-GalpNAc-(1→3)-[α-L-Fucp-(1→2)]-β-D-Galp-(1→3)-β-D-GlcpNAc-R,
A type II α-D-GalpNAc-(1→3)-[α-L-Fucp-(1→2)]-β-D-Galp-(1→4)-β-D-GlcpNAc-R,
A type III α-D-GalpNAc-(1→3)-[α-L-Fucp-(1→2)]-β-D-Galp-(1→3)-α-D-GalpNAc-R,
A type IV α-D-GalpNAc-(1→3)-[α-L-Fucp-(1→2)]-β-D-Galp-(1→3)-β-D-GalpNAc-R,
A type V α-D-GalpNAc-(1→3)-[α-L-Fucp-(1→2)]-β-D-Galp-(1→3)-β-D-Galp-R,
A type VI α-D-GalpNAc-(1→3)-[α-L-Fucp-(1→2)]-β-D-Galp-(1→4)-β-D-Glcp-R,
B type I α-D-Galp-(1→3)-[α-L-Fucp-(1→2)]-β-D-Galp-(1→3)-β-D-GlcpNAc-R,
B type II α-D-Galp-(1→3)-[α-L-Fucp-(1→2)]-β-D-Galp-(1→4)-β-D-GlcpNAc-R,
B type III α-D-Galp-(1→3)-[α-L-Fucp-(1→2)]-β-D-Galp-(1→3)-α-D-GalpNAc-R,
B type IV α-D-Galp-(1→3)-[α-L-Fucp-(1→2)]-β-D-Galp-(1→3)-β-D-GalpNAc-R,
B type V α-D-Galp-(1→3)-[α-L-Fucp-(1→2)]-β-D-Galp-(1→3)-β-D-Galp-R,
B type VI α-D-Galp-(1→3)-[α-L-Fucp-(1→2)]-β-D-Galp-(1→4)-β-D-Glcp-R,
H type I α-L-Fucp-(1→2)-β-D-Galp-(1→3)-β-D-GlcpNAc-R,
H type II α-L-Fucp-(1→2)-β-D-Galp-(1→4)-β-D-GlcpNAc-R,
H type III α-L-Fucp-(1→2)-β-D-Galp-(1→3)-α-D-GalpNAc-R,
H type IV α-L-Fucp-(1→2)-β-D-Galp-(1→3)-β-D-GalpNAc-R,
H type V α-L-Fucp-(1→2)-β-D-Galp-(1→3)-β-D-Galp-R, and
H type VI α-L-Fucp-(1→2)-β-D-Galp-(1→4)-β-D-Glcp-R,
wherein R is a linker group for covalently attaching the antigen subtypes to the microarray or macroarray.

42. The assay kit of any one of claims 38-41 additionally comprising one or more of:
(i) a container of wash solution; and
(ii) a container of a labeled secondary antibody specific for binding complexes formed from the anti-ABO antigen subtype antibodies and the immobilized antigens.

43. A method of identifying the ABO histo-blood subgroup of a subject comprising:
(a) determining an anti-ABO histo-blood group antigen subtype antibody profile using a biological sample from said subject; and
(b) comparing the determined antibody profile to known ABO histo-blood group antigen subtype profiles and/or known anti-ABO antigen subtype antibody profiles for ABO histo-blood subgroups to identify the ABO histo-blood subgroup of the subject.

44. The method of claim 43, wherein the step of determining the anti-ABO histo-blood group antigen subtype antibody profile comprises:
(a) applying the biological sample from the subject to a glycan microarray or macroarray containing a plurality of immobilized ABO antigen;
(b) incubating the biological sample with the glycan microarray or macroarray such that binding complexes form between anti-ABO histo-blood group antigen subtype antibodies present in the sample and the bound antigens; and
(c) detecting the binding complexes and identifying corresponding antigens.

45. The method of claim 44, wherein the immobilized ABO antigen subtypes are selected from the group consisting of:
A type I α-D-GalpNAc-(1→3)-[α-L-Fucp-(1→2)]-β-D-Galp-(1→3)-β-D-GlcpNAc-R,
A type II α-D-GalpNAc-(1→3)-[α-L-Fucp-(1→2)]-β-D-Galp-(1→4)-β-D-GlcpNAc-R,
A type III α-D-GalpNAc-(1→3)-[α-L-Fucp-(1→2)]-β-D-Galp-(1→3)-α-D-GalpNAc-R,
A type IV α-D-GalpNAc-(1→3)-[α-L-Fucp-(1→2)]-β-D-Galp-(1→3)-β-D-GalpNAc-R,
A type V α-D-GalpNAc-(1→3)-[α-L-Fucp-(1→2)]-β-D-Galp-(1→3)-β-D-Galp-R,
A type VI α-D-GalpNAc-(1→3)-[α-L-Fucp-(1→2)]-β-D-Galp-(1→4)-β-D-Glcp-R,
B type I α-D-Galp-(1→3)-[α-L-Fucp-(1→2)]-β-D-Galp-(1→3)-β-D-GlcpNAc-R,
B type II α-D-Galp-(1→3)-[α-L-Fucp-(1→2)]-β-D-Galp-(1→4)-β-D-GlcpNAc-R,
B type III α-D-Galp-(1→3)-[α-L-Fucp-(1→2)]-β-D-Galp-(1→3)-α-D-GalpNAc-R,
B type IV α-D-Galp-(1→3)-[α-L-Fucp-(1→2)]-β-D-Galp-(1→3)-β-D-GalpNAc-R,
B type V α-D-Galp-(1→3)-[α-L-Fucp-(1→2)]-β-D-Galp-(1→3)-β-D-Galp-R,
B type VI α-D-Galp-(1→3)-[α-L-Fucp-(1→2)]-β-D-Galp-(1→4)-β-D-Glcp-R,
H type I α-L-Fucp-(1→2)-β-D-Galp-(1→3)-β-D-GlcpNAc-R,
H type II α-L-Fucp-(1→2)-β-D-Galp-(1→4)-β-D-GlcpNAc-R,
H type III α-L-Fucp-(1→2)-β-D-Galp-(1→3)-α-D-GalpNAc-R,
H type IV α-L-Fucp-(1→2)-β-D-Galp-(1→3)-β-D-GalpNAc-R,
H type V α-L-Fucp-(1→2)-β-D-Galp-(1→3)-β-D-Galp-R, and
H type VI α-L-Fucp-(1→2)-β-D-Galp-(1→4)-β-d-Glcp-R,
wherein R is a linker group for covalently attaching the antigen subtypes to the microarray or macroarray.

46. The method of claim 44 or 45, wherein the step of detecting the binding complexes comprises:
(i) incubating the binding complexes immobilized on the glycan microarray or macroarray with a labeled secondary antibody that binds to the binding complexes and/or the bound anti-ABO histo-blood group antigen subtype antibodies; and
(ii) detecting the bound labeled secondary antibody.
